(19) United States
(12) Patent Application Publication
(10) Pub. No.: US 2016/0216276 A1
(43) Pub. Date: Jul. 28, 2016

(54) SERUM ANTIBODY ASSAY FOR DETERMINING PROTECTION FROM MALARIA, AND PRE-ERYTHROCYTIC SUBUNIT VACCINES

(71) Applicants: Sanaria Inc., Rockville, MD (US); Antigen Discovery, Inc., Irvine, CA (US); The United States of America,as represented by the Secretary, Department of Health and Human Service, Bethesda, MD (US)

(72) Inventors: Philip FELGNER, Irvine, CA (US); Stephen L. HOFFMAN, Rockville, MD (US); Robert SEDER, Bethesda, MD (US)

(21) Appl. No.: 14/914,869

(22) PCT Filed: Sep. 2, 2014
(86) PCT No.: PCT/US2014/053745

§ 371 Date: Feb. 26, 2016

Related U.S. Application Data
(60) Provisional application No. 61/872,527, filed on Aug. 30, 2013.

(51) Int. Cl. G01N 33/68; A61K 39/015

(52) U.S. Cl. CPC G01N 33/6854; A61K 39/015

(57) ABSTRACT

Disclosed herein are diagnostic assays for identifying individuals that are protected against Plasmodium falciparum caused malaria. Such assays are particularly useful for determining not only the protective efficacy of Pf whole parasite vaccines for individual subjects, but also within populations of vaccinated subjects. The assays comprise the use of proteomes representing at least 50% of Pf, preferably coupled to a solid phase as a fixed array. The arrays are used to probe the sera of human subjects, particularly subjects of human clinical trials of whole parasite malaria vaccines as well as public health vaccination campaigns. Serum samples with antibody profiles most strongly reactive in multiplex to CSP and MSP5 demonstrate a sensitivity of from 92% to 100% and a specificity of from 84% to 89%.

### FIELD OF THE INVENTION

This invention relates generally to the field of malaria immunology. More particularly, it relates to novel approaches to the identification and use of particular combinations of humoral antibodies diagnostic of malaria protection, including protection induced by vaccination, and diagnostic assays derived therefrom. The invention also relates to the identification of combinations of P. falciparum antigens useful as multi-component subunit vaccines.

### REFERENCE TO A SEQUENCE LISTING SUBMITTED ELECTRONICALLY VIA EFS-WEB

The content of the electronically submitted sequence listing (Name: “2602_012PC01_SequenceListing_ascii”; Size: 31,833 bytes; and Date of Creation: Aug. 29, 2014) filed herewith with the application is incorporated by reference in its entirety.

### BACKGROUND OF THE INVENTION

Malaria control interventions, including insecticide-impregnated bednets, insecticide spraying, and antimalarial drugs, have reduced malaria morbidity and mortality substantially (World Health Organization, World Malaria Report: 2012 (2012; www.who/int/malaria/publications/world_malaria_report 2012/report/en/index.html). However, in 2010, despite these measures, there were an estimated 220 million clinical cases and 0.66 to 1.24 million deaths caused by malaria (World Health Organization, World Malaria Report: 2012, C. J. Murray et al., Lancet 379, 413-431 (2012)). A highly effective vaccine will be ideal for preventing malaria in individuals and eliminating malaria in defined geographic areas. It would optimally target the parasite at asymptomatic, pre-erythrocytic stages (C. V. Plowe et al., J. Infect. Dis. 200, 1646-1649 (2009), malERA Consultative Group on Vaccines, A research agenda for malaria eradication: vaccines. PLoS Med. 8, e1000398 (2011)). The World Health Organization malaria vaccine technology roadmap set a vaccine efficacy goal of 80% by 2025 (Malaria Vaccine Technology Roadmap, 2006; www.malariavaccine.org/files/Malaria_Vaccine_TRM_Final.pdf). Heretofore, no injectable malaria vaccine candidate has consistently approached that level of efficacy.

RTS,S/AS01 is the most advanced subunit malaria vaccine, protecting approximately 50% of adult subjects against controlled human malaria infection (hereinafter, “CHMI”) 2-3 weeks after the last dose of immunization (K. E. Kester et al., J. Infect. Dis. 200, 337-346 (2009)). In a large-scale phase 3 trial in African infants 6-12 weeks of age, RTS,S/AS01 reduced the rates of clinical and severe malaria acquired over a 12-month period by 31.3% and 36.6%, respectively (S. T. Agnandji et al., N. Engl. J. Med. 367, 2284-2295 (2012)).

On the other hand, it has been known for over 40 years that high-level, enduring protective immunity can be provided by means of the bites of >1000 mosquitoes, infected with radiation attenuated Plasmodium falciparum (Pf) sporozoites (SPZ) (R. S. Nussenzweig, et al., Nature 222, 488-489 (1969); D. F. Clyde, et al., Am. J. Med. Sci. 266, 169-177 (1973); K. H. Rieckmann, et al., Trans. R. Soc. Trop. Med. Hyg. 68, 258-259 (1974); S. L. Hoffman, et al., J. Infect. Dis. 185, 1155-1164 (2002)). As a practical matter, this approach to malaria vaccination required the capacity to manufacture live, aseptic, radiation-attenuated, purified, preserved PfSPZ as the immunogen of an injectable vaccine that meets regulatory standards (T. C. Luke, et al., J. Exp. Biol. 206, 3803-3808 (2003); S. L. Hoffman, et al., Hum. Vaccin. 6, 97-106 (2010); J. E. Epstein, et al., Science 334, 475-480 (2011)).

PfSPZ Vaccine—

The first clinical trial of PfSPZ Vaccine, comprising the Pf NF54 strain of SPZ (T. Ponnudurai, et al., Trans. R. Soc. Trop. Med. Hyg. 76, 242-250 (1982)) was conducted in 80 immunologically naïve adults (T. C. Luke, et al., (2003)). They received up to 6 doses of 1.35×10⁵ SPZ subcutaneously (SC) or intradermally (ID). PfSPZ Vaccine proved safe and well-tolerated, but elicited low-level immune response and minimal protection. It was hypothesized that the limited efficacy was due to the inefficiency of the ID and SC routes of administration (S. Chakravarty, et al., Nat. Med. 13, 1035-1041 (2007)). Subsequent studies in non-human primates (NHP) with the PfSPZ Vaccine showed that IV, but not SC, administration elicited potent and durable PfSPZ-specific T-cell responses in peripheral blood, and most notably in the liver (T. C. Luke, et al., (2003)), the likely site of immune protection (S. L. Hoffman, et al., Nat. Med. 6, 1218-1219 (2000)).

Based on these results, a phase 1 clinical trial was conducted to determine safety, immunogenicity and protective efficacy of IV administration of PfSPZ Vaccine (Seder, R. A., et al., Science, 341:1359-1365 (2013; incorporated herein by reference in its entirety). PfSPZ Vaccine-induced protection against Pf malaria was safe, well tolerated and highly protective when administered up to 6 times IV to 40 adults. Six of six adult subjects receiving 6.75×10⁵ SPZ in 5 doses were protected as were 6 of 9 adult subjects who received 5.4×10⁵ SPZ in 4 doses (Seder, R. A., et al.).

Vaccines can also be genetically attenuated (Janse, C., et al., WO/2014/116990 (2014)). In another approach, infectious PfSPZ are administered in the presence of an antimalarial prophylactic such as chloroquine (Roestenberg, M., et al., Lancet, 377:1770-1776 (2011)).

Controlled Human Malaria Infection (CHMI)—

The evaluation of the efficacy of all malaria vaccines and treatments benefits from the fact that for subjects in a clinical study, careful monitoring results in early diagnosis of the onset of the signs, symptoms and pathology of malaria and its rapid and effective treatment. Thus, CHMI has become a definitive method by which the efficacy of vaccination against malaria can be measured. In the trial described in Seder et al. challenge, CHMI was achieved by means of exposing subjects to Pf-infected mosquitoes that met standard infectivity criteria (K. E. Kester et al., (2009); T. C. Luke, et al., (2003); J. D. Chulay, et al. Am. J. Trop. Med. Hyg. 35, 66-68 (1986); L. S. Rickman, et al. Am. J. Trop. Med. Hyg. 43, 441-445 (1990); K. E. Lyke, et al., PLoS ONE 5, e13490 (2010)) approximately 3 weeks after the last dose of PfSPZ Vaccine. The mosquitoes used were infected with Pf NF54 or a clone thereof. Subjects were monitored as outpatients for 7 days after CHMI and then admitted to the NIH Clinical Center for up to 11 consecutive nights or until diagnosis and cure of parasitemia was documented. Daily thick blood smears were performed, with additional smears performed when needed, based on clinical presentation. Treatment with chloroquine or atovaquone/proguanil was initiated when a thick blood smear had ≧2 Pf parasites in 0.5 μL blood, confirmed by an expert malaria microscopist. Subjects were discharged after two consecutive days with negative malaria smears or on post-CHMI day 18 if not parasitemic. Subjects were considered protected if smears were negative through day 28 post-CHMI. Quantitative PCR (qPCR) was performed to detect parasite DNA in blood.

While effective, CHMI is cumbersome and expensive, requiring participation of several clinicians, experts, and hospital facilities. Clearly, a diagnostic assay that correlates well with clinical observation would be very useful in the pursuit of protective vaccines, and treatments of malaria.

### SUMMARY OF THE INVENTION

A clinical trial testing the immunogenicity and efficacy of aseptic, radiation-attenuated, purified, cryopreserved PfSPZ sporozoites used as the immunogen (PfSPZ Vaccine, provided by Sanaria Inc.), and administered by intravenous injection, resulted in 13 individuals that were protected against controlled human malaria infection (CHMI) and 19 that were not protected. In the highest dose group 6 out of 6 individuals were protected and the next lower dose group had 6 out of 9 protected (Seder, et al. Science 2013). The serum specimens from the subjects of this trial provided an opportunity to determine immune parameters, particularly the P falciparum-specific antibody profile associated with the vaccine-mediated protective response. A microarray was constructed containing 4,528 P. falciparum (Pf) protein features representing 50% of the parasite proteome, and it was probed with the serum specimens from this trial.

One hundred percent of the sera from protected subjects and 23% from unprotected subjects had significant antibody levels against the Pf circumsporozoite protein (CSP). Seventy seven percent of sera from protected subjects and 0% from unprotected had antibodies against MSP5. Depending on the statistical treatment, CSP and MSP5 used together in a multiplex serodiagnostic assay predicted protection from malaria with 92% to 100% sensitivity (positive results for protected individuals) and 84% to 89% specificity (negative results for unprotected individuals). Thus, when used in concert as a multiplex serodiagnostic assay, antibody responses to CSP and MSP5 or antigenic fragments thereof are an accurate biomarker of PfSPZ vaccine-mediated protection against malaria.

Provided herein is a method for determining a state of protective immunity against P. falciparum-induced malaria in a human subject said method comprising probing a body fluid sample of said subject with Pf immunological determinants wherein said fluid comprises antibodies and at least two of said Pf immunological determinants are encoded by CSP encoding nucleic acids (SEQ ID NO:1) and MSP5 encoding nucleic acids (SEQ ID NO:2) or are antigenic fragments of the polypeptides encoded by nucleic acids SEQ ID NO:1 and SEQ ID NO:2 and wherein said the immunological determinants specifically bind antibodies. In certain embodiments, the Pf immunological determinants also include polypeptides encoded by one or more of nucleic acid sequences SEQ ID NO:3-8 or antigenic fragments thereof. In certain embodiments, the antibody/immunological determinant complexes are detected.

Further provided is a subunit vaccine comprising immunologic determinants encoded by CSP nucleic acids (SEQ ID NO: 1) and MSP5 encoding nucleic acids (SEQ ID NO:2) or antigenic fragments of the polypeptides encoded by nucleic acids SEQ ID NO:1 and SEQ ID NO:2. While CSP per se is already a well-recognized target antigen for pre-erythrocytic stage malaria vaccine development (RTS,S/AS01), and Pf MSP5 has been disclosed (See, e.g., U.S. Pat. No. 8,026,354), the results disclosed herein demonstrate that a subunit vaccine comprising both CSP and MSP5 in concert will provide a protective human humoral response against pre-erythrocytic stages of P. falciparum infection, as well as infection from other Plasmodium species parasites.

### DESCRIPTION OF THE FIGURES

FIG. 1 shows a heat map derived from the sera of individual subjects in protected and unprotected groups and receiving low (1), medium (m), or high (h) dosages of PfSPZ vaccine and examining the top 50 differentially reactive antigens.

FIG. 2 shows thirty-two sera samples from the intravenous injection immunization trial of PfSPZ Vaccine probed with Pf1000 microarray down-selected from a large array containing 4,528 Plasmodium falciparum (Pf) protein features representing 50% of the parasite proteome. Among the 32 sera samples, 13 were obtained from the protected individuals and 19 from the unprotected individuals. The graph shows the top 50 differentially reactive antigens when compared the protected and unprotected groups. The graph is sorted by the antigen reactivity of protected group along with the p-value and cut-off value. A and B differ only in the scale of reactivity.

FIG. 3 shows the reactivity of: CSP (PFC0210c) (encoded by SEQ ID NO:1); MSP5 (PFB0305c_1o2) (encoded by SEQ ID NO:2); and MSP5 (PFB0305c_e1) (encoded by SEQ ID NO:2).

FIG. 4 shows the reactivity of: SNARE proteins, putative (SYN6) (PFE1505w_2o2) (encoded by SEQ ID NO:3); Plasmodium exported protein (hyp2), unknown function (PF10_0024_2o2) (encoded by SEQ ID NO:4); and Conserved Plasmodium protein, unknown function (PF10_0295_1o1) (encoded by SEQ ID NO:5).

FIG. 5 shows the reactivity of: Histone-lysine-N-methyltransferase, H3 lysine-4-specific (SET10) (PFL1010ce1s2) (encoded by SEQ ID NO:6); Eukaryotic translation initiation factor eIF2A, putative (PF14_0360e1s1) (encoded by SEQ ID NO:7); and Pre-mRNA-splicing helicase BRR2, putative (BRR2) (PFD1060we1s1) (encoded by SEQ ID NO:8).

FIG. 6A-I shows ROC statistical analysis graphs of protected versus unprotected groups in top 9 sero-reactive antigens: A) CSP (PFC0210c) (encoded by SEQ ID NO:1); B) MSP5 (PFB0305c_1o2) (encoded by SEQ ID NO:2); C) MSP5 (PFB0305c_e1) (encoded by SEQ ID NO:2); D) PFE1505w_2o2 (encoded by SEQ ID NO:3); E) PF10_0024_2o2 (encoded by SEQ ID NO:4); F) PF10_0295_1o1 (encoded by SEQ ID NO:5); G) PFL1010ce1s2 (encoded by SEQ ID NO:6); H) PF14_0360e1s1 (encoded by SEQ ID NO:7); and I) PF14_0360e1s1 (encoded by SEQ ID NO:7), along with AUC values.

FIG. 7A-F shows further ROC analysis is performed by combining reactivity of MSP5 and CSP via various summation or multiplication: A) CSP, B) MSP5, C) Multiply MSP5 and CSP, D) Sum MSP & CSP, E) Sum 10×MSP5 & CSP, and F) Sum 100×MSP5 & CSP.

### DETAILED DESCRIPTION OF THE INVENTION

### Definitions

As used herein, “sensitivity” means the differential formation of complexes comprising an antigen binding molecule, e.g., antibodies in human body fluids, and a Pf immunologic determinant (positive reaction) in individuals having protective immunity against malaria infection.

As used herein, “specificity” means the lack of differential formation of complexes comprising an antigen binding molecule, e.g., antibodies in human body fluids, and a Pf immunologic determinant (negative reaction) in individuals that are determined to lack protective immunity against malaria infection.

As used herein, “lacking” or to “lack” means being deficient in, or not having a sufficient amount.

As used herein, “immunologic determinant” means an antigen, an antigenic epitope, or an sero-reactive peptide or protein.

As used herein, “differential reactivity” is a comparison of the immunoreactivities of an immunologic determinant with an antigen binding molecule, e.g., antibodies in the human body fluids, of protected and unprotected subjects, wherein p-values are less than 0.05 as calculated using two-tailed Student's t-test of unequal variance.

As used herein an immunologic determinant “encoded by a nucleic acid sequence” means those nucleic acid sequences are transcribed to mRNA, which is translated into the polypeptides which are the immunologic determinants.

As used herein “probing” a human body fluid means exposing that body fluid to one or more immunologic determinants and measuring the specific reactivity of the immunologic determinants to an antigen binding molecule, e.g., antibodies, in the human body fluid. As used herein, this measurement is referred to as an “immunoreactivity determination”.

As used herein a “detection agent” is a molecule or a combination of molecules that specifically recognizes the complex formed by the binding of an immunologic determinant to an antigen binding molecule.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide or antigenic fragment thereof of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure.

By an “isolated” polypeptide or a fragment, variant, or derivative thereof is intended a polypeptide that is not in its natural milieu. Recombinantly produced polypeptides and proteins expressed in host cells are considered isolated for purpose of the invention, as are native or recombinant polypeptides that have been separated, fractionated, or partially or substantially purified by any suitable technique.

Also included as polypeptides of the present invention are fragments, derivatives, analogs, or variants of the foregoing polypeptides, and any combination thereof. The terms “fragment,” “variant,” “derivative,” and “analog” polypeptides of the present application include any polypeptides that retain at least some of the properties of the corresponding polypeptide of the application. Fragments of polypeptides of the present invention include proteolytic fragments, as well as deletion fragments, in addition to specific antibody binding fragments discussed elsewhere herein. Variant polypeptides of the present application include fragments and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions, or additions. Variant polypeptides may also be referred to herein as “polypeptide analogs.” As used herein a “derivative” of a polypeptide refers to a subject polypeptide having one or more residues chemically derivatized by reaction of a functional side group. Also included as “derivatives” are those peptides that contain one or more naturally occurring amino acid derivatives of the twenty standard amino acids. Derivatives of polypeptides of the present application may include polypeptides that have been altered so as to exhibit additional features not found on the reference polypeptide of the application.

The term “polynucleotide” is intended to encompass nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, that has been removed from its native environment. For example, a recombinant polynucleotide encoding polypeptide or antigenic fragment thereof contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

As used herein, a “coding region” is a portion of nucleic acid that consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding a polypeptide or antigenic fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A “binding molecule” or “antigen binding molecule” of the present application refers in its broadest sense to a molecule that specifically binds an antigenic determinant of an antigen.

As used herein “immunoreactive” means the binding of an immunologic determinant with an antigen binding molecule. When immunoreactivity occurs with immunologic determinants of human serum this is also referred to as “sero-reactive”.

In certain embodiments, “specific immunoreactivity” refers to reacting to a particular Pf immunologic determinant.

In certain embodiments, as used herein, the term “about” means plus or minus 5% of the numerical value of the number with which it is being used. Therefore, about 85% means in the range of 82.5% to 87.5% as described herein.

In some embodiments, a binding molecule of the invention is an antibody or an antigen binding fragment thereof.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al. (1988) Antibodies: A Laboratory Manual (2nd ed.; Cold Spring Harbor Laboratory Press).

The portion of an antigen or polypeptide that specifically interacts with the antigen binding domain of an antibody is an “epitope,” or an “antigenic determinant.” An antigen or polypeptide may comprise a single epitope, but typically comprises at least two epitopes, and can include any number of epitopes, depending on the size, conformation, and type of antigen.

The term “antigenic fragment” and “antibody binding fragment” are used interchangeably herein. An antigenic fragment as used herein is able to complex with the same antigen binding molecule, e.g., antibody in human body fluid, as the immunogenic determinant from which it is derived.

“Conferring protective immunity” as used herein refers to providing to a population or a subject (i.e., an individual) the ability to generate an immune response to protect against a disease (e.g., malaria) caused by subsequent exposure to a pathogen (e.g., Plasmodium falciparum) such that the clinical manifestations, pathology, or symptoms of disease are reduced during subsequent exposure to the pathogen as compared to a non-treated host, or such that the rate at which infection, or clinical manifestations, pathology, or symptoms of disease appear within a population are reduced, as compared to a non-treated population.

“Immune response” as used herein means a response in the recipient to the introduction of attenuated sporozoites generally characterized by, but not limited to, production of antibodies and/or T cells. Generally, an immune response may be a cellular response such as induction or activation of CD4+ T cells or CD8+ T cells specific for Plasmodium-species epitopes, a humoral response of increased production of Plasmodium-specific antibodies, or both cellular and humoral responses. With regard to a malaria vaccine, the immune response established by a vaccine comprising sporozoites includes but is not limited to responses to proteins expressed by extracellular sporozoites or other stages of the parasite after the parasites have entered host cells, especially hepatocytes and mononuclear cells such as dendritic cells and/or to components of said parasites. In the instant invention, upon subsequent challenge by infectious organisms, the immune response prevents development of pathogenic parasites to the asexual erythrocytic stage that causes disease.

“Vaccine” as used herein is a preparation comprising an immunogenic agent and a pharmaceutically acceptable diluent potentially in combination with excipient, adjuvant and/or additive or protectant. The immunogen may be comprised of a whole infectious agent or a molecular subset of the infectious agent (produced by the infectious agent, synthetically or recombinantly). When the vaccine is administered to a subject, the immunogen stimulates an immune response that will, upon subsequent challenge with infectious agent, protect the subject from illness or mitigate the pathology, symptoms or clinical manifestations caused by that agent. A therapeutic (treatment) vaccine is given after infection and is intended to reduce or arrest disease progression. A preventive (prophylactic) vaccine is intended to prevent initial infection or reduce the rate or burden of the infection. Agents used in vaccines against a parasitic disease such as malaria may be whole-killed (inactive) parasites, live-attenuated parasites (unable to fully progress through their life cycle), or purified or artificially manufactured molecules associated with the parasite—e.g. recombinant proteins, synthetic peptides, DNA plasmids, and recombinant viruses or bacteria expressing Plasmodium proteins. A vaccine may comprise sporozoites along with other components such as excipient, diluent, carrier, preservative, adjuvant or other immune enhancer, or combinations thereof, as would be readily understood by those in the art.

As used herein, “inoculate” means to administer a clinically relevant dose of a vaccine. In some embodiments, the vaccine is a P. falciparum vaccine, e.g., a P. falciparum whole parasite vaccine.

Certain embodiments of the application are directed to a method for determining a state of protective immunity against P. falciparum-induced malaria in a human subject said method comprising probing a body fluid sample of said subject with Pf immunologic determinants comprising wherein said body fluid sample comprises antibodies and two of said Pf immunologic determinants used are selected from the group consisting of polypeptides encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof, and wherein said antibodies are specifically immunoreactive to the Pf antigens.

In certain embodiments of the application, the polypeptide is encoded by the nucleic acid sequence at least 80%, 85%, 95%, 99%, or 100% identical to SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or is an antigenic fragment thereof. In certain embodiments, two Pf immunologic determinants are encoded by nucleic acid sequences at least 80%, 85%, 95%, 99%, or 100% identical to SEQ ID NO:1 and SEQ ID NO:2, or antigenic fragments thereof.

Certain embodiments of the application are directed to a method for determining a state of protective immunity against P. falciparum-induced malaria in a human subject said method comprising probing a body fluid sample of said subject with Pf immunologic determinants comprising: (i) a first immunologic determinant related to Circumsporozoite Protein (CSP) or a fragment thereof that is at least 80%, 85%, 90%, 95%, 99%, or 100% identical to an amino acid sequence encoded by the nucleic acid sequence of SEQ ID NO: 1; and (ii) a second immunologic determinant related to Merozoite Surface Protein 5 (MSP5) or a fragment thereof that is at least 80%, 85%, 90%, 95%, 99%, or 100% identical to an amino acid sequence encoded by the nucleic acid sequence of SEQ ID NO:2, and wherein said antibodies are specifically immunoreactive to the Pf immunologic determinants.

Certain embodiments of the application are directed to a method for identifying protective immunity against P. falciparum-induced malaria in a human subject, wherein the protective immunity is identified by detection of antibodies specific to immunologic determinants of at least two Pf immunologic determinants coupled to a solid surface, wherein the method comprises:

(i) applying the body fluid sample from said subject to said solid surface, said solid surface comprising (a) a polypeptide having a sequence that is at least 80%, 85%, 90%, 95% or 100% identical to an amino acid sequence encoded by the nucleic acid sequence of SEQ ID NO:1 or an antigenic fragment thereof and (b) a polypeptide having a sequence that is at least 80%, 85%, 90%, 95% or 100% identical to an amino acid sequence encoded by the nucleic acid sequence of SEQ ID NO:2 or an antigenic fragment thereof;

(ii) applying a detection agent that binds antibody-Pf immunologic determinant complexes to the solid surface of (i); and

(iii) identifying protective immunity against P. falciparum-induced malaria in said subject by detecting antibody binding to the polypeptide of (a) and the polypeptide of (b), by means of determining the presence of the detection agent of step (iii).

In some embodiments, step (i) further comprises applying one or more of the polypeptides having a sequence that is at least 80%, 85%, 90%, 95% or 100% identical to an amino acid sequence encoded by the nucleic acid sequence of SEQ ID NO:3, SEQ ID NO:4, SEQ ID NO:5, SEQ ID NO:6, SEQ ID NO:7, or SEQ ID NO:8, or antigenic fragments thereof.

Certain embodiments of the application are directed to a method for identifying protective immunity against P. falciparum-induced malaria in a human subject by a means for detecting the presence of antibodies present in a body fluid sample that specifically bind to particular antigens of P. falciparum, comprising contacting the sample with (i) a polypeptide having a sequence that is at least 80%, 85%, 90%, 95%, 99% or 100% identical to an amino acid encoded by a polynucleotide comprising SEQ ID NO:1 or an antigenic fragment thereof and (ii) a polypeptide having a sequence that is at least 80%, 85%, 90%, 95%, 99% or 100% identical to an amino acid encoded by a polynucleotide comprising SEQ ID NO:2 or an antigenic fragment thereof, and detecting antibody-immunologic determinant complexes. Some embodiments comprise further contacting the sample with one or more polypeptides encoded by the nucleic acid sequence of SEQ ID NO:3, SEQ ID NO:4, SEQ ID NO:5, SEQ ID NO:6, SEQ ID NO:7, or SEQ ID NO:8, or antigenic fragments thereof, and determining the presence of further antibody/Pf immunologic determinant complexes.

Certain embodiments of the application are directed to methods for identifying a human subject with protective immunity against P. falciparum-induced malaria by a means for detecting antibodies that specifically bind to immunologic determinants of P. falciparum present in a body fluid sample, comprising analyzing the body fluid sample for detection of antibodies that specifically immunoreact with particular Pf immunologic determinants or with polypeptides having a sequence that is at least 80%, 85%, 90%, 95%, 99% or 100% identical to an amino acid encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof, and wherein the subject is identified as having protective immunity against P. falciparum-induced malaria if the antibodies that specifically immunoreact with the Pf immunologic determinants are detected or if antibody-Pf immunologic determinant complexes are found.

Certain embodiments of the application are directed to a method for identifying a human subject lacking protective immunity against P. falciparum-induced malaria and in need of said protective immunity, comprising analyzing a body fluid sample of a subject for the presence or absence of antibodies that specifically immunoreact with particular Pf immunologic determinants, wherein said Pf immunologic determinants are selected from the group consisting of polypeptides having a sequence that is at least 80%, 85%, 90%, 95%, 99% or 100% identical to an amino acid encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof, wherein subjects having samples lacking antibodies that specifically immunoreact with the Pf immunologic determinants are identified as lacking protective immunity against P. falciparum-induced malaria. In certain embodiments, the subjects lacking protective immunity are inoculated with a P. falciparum vaccine.

Certain embodiments of the application are directed to conferring protective immunity against P. falciparum-induced malaria in subjects in need of such protective immunity, comprising requesting a test providing the analysis of a body fluid sample of a subject for detection of the presence or absence of antibodies that specifically immunoreact with particular Pf immunologic determinants, wherein said Pf immunologic determinants are selected from the group consisting of polypeptides having a sequence that is at least 80%, 85%, 90%, 95%, 99% or 100% identical to an amino acid encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof, and subjects having samples with antibodies that do not specifically immunoreact with the Pf immunologic determinants are then inoculated with a P. falciparum vaccine.

In some embodiments, said Pf antigens consist of polypeptides having a sequence that is at least 80%, 85%, 90%, 95%, 99% or 100% identical to: (i) Circumsporozoite Protein (CSP) encoded by the nucleic acid sequence of SEQ ID NO:1 or an antigenic fragment thereof and (ii) Merozoite Surface Protein 5 (MSP5) encoded by the nucleic acid sequence of SEQ ID NO: 2 or an antigenic fragment thereof.

In some embodiments the body fluid sample is serum or whole blood. In some embodiments, the serum is collected no sooner than 3, 4, 5, 6, 7, 8, 9, 10, 11, 12, 13 or 14 days after the last dose of vaccine, e.g., between 3-21 days, 7-21 days, 7-14 or 10-14 days (e.g., 14 days) after the last dose of a vaccine, e.g., a P. falciparum whole parasite vaccine.

In some embodiments, the method is an in vitro assay for detecting antibodies. In some embodiments, the subject has first been inoculated with a P. falciparum vaccine. In some embodiments, the vaccine is administered in multiple doses. In some embodiments, the antigen, e.g., a Pf antigen (e.g., polypeptides having a sequence that is at least 80%, 85%, 90%, 95%, 99% or 100% identical to an amino acid sequence encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragment thereof) is detectably labeled. In some embodiments, the antigens or antigenic fragments are attached to a surface, e.g., a solid surface. In some embodiments, the antigens are provided as an array coupled to a solid phase. In some embodiments, the antibody immunoreactivity is determined by enzyme-linked immunosorbent assay (ELISA).

In some embodiments, the method has a sensitivity of at least 80% to 100%, 85%-100%, or 90%-100%. In some embodiments, the determination has a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, or 100%.

In some embodiments, the method has a specificity of at least 80% to 100%, 85%-100%, or 90%-100%. In some embodiments, the determination has a specificity of at least about 80%, 81%, 82%, 83%, 84%, 85%, 86%, 87%, 88%, 89%, 90%, 91%, 92%, 93%, 94%, 95%, 96%, 97%, 98%, 99%, or 100%.

In certain embodiments, the sensitivity and/or specificity of the method is improved by determining or detecting binding of at least two, at least three, at least four, at least five, at least six, at least seven, or at least eight antibodies which bind to at least two polypeptides that are at least 80%, 85%, 90%, 95%, 99% or 100% identical to amino acid sequences encoded by the nucleic acid sequences SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof. In some embodiments, the polypeptides are encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof.

Certain embodiments of the application are directed to a subunit vaccine protective against P. falciparum-caused malaria comprising two or more Pf antigens that are selected from the group consisting of polypeptides encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof.

Certain embodiments of the application are directed to a subunit vaccine protective against P. falciparum-caused malaria comprising (i) a polypeptide encoded by a nucleic acid sequence comprising SEQ ID NO:1 or an antigenic fragment thereof and (ii) a polypeptide encoded by a nucleic acid sequence comprising SEQ ID NO:2 or an antigenic fragment thereof.

In some embodiments the vaccine further comprises one or more of the polypeptides encoded by a nucleic acid sequence selected from the group consisting of SEQ ID NO:3, SEQ ID NO:4, SEQ ID NO:5, SEQ ID NO:6, SEQ ID NO:7, or SEQ ID NO:8, or antigenic fragments thereof.

Some embodiments are directed to a whole parasite vaccine further comprising two or more Pf antigens that are selected from the group consisting of polypeptides encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof.

In some embodiments the vaccine is a whole parasite vaccine further comprising (i) a polypeptide encoded by a nucleic acid sequence comprising SEQ ID NO:1 or an antigenic fragment thereof and (ii) a polypeptide encoded by a nucleic acid sequence comprising SEQ ID NO:2 or an antigenic fragment thereof.

In some embodiments the Pf antigens that are selected from the group consisting of polypeptides encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof are exposed to serum samples of the test subjects. In other embodiments, the Pf antigens that are selected from the group consisting of polypeptides encoded by the nucleic acid sequence SEQ ID NO:1, SEQ ID NO:2, SEQ ID NO:3, SEQ ID NO:4, SEQ ID NO:5, SEQ ID NO:6, SEQ ID NO:7, or SEQ ID NO:8, or antigenic fragments thereof are exposed to blood samples of the test subjects.

In some embodiments, the polypeptide of the application has a sequence that is at least 80%, at least 85%, at least 90%, at least 95%, at least 99%, or 100% identical to an amino acid sequence encoded by SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In some embodiments, the polynucleotide of the application has a sequence that is at least 80%, at least 85%, at least 90%, at least 95%, at least 99%, or 100% identical to SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

The method provided herein is based on the identification of immunodominant Plasmodium falciparum (Pf) antigens appearing differentially in the body fluids, particularly sera, of protected vs. unprotected subjects that have been previously exposed to Pf infection, either as a result of natural exposure or intentionally as a result of protective vaccination. A similar approach to the identification of immunodominant antigens resulting from pathogenic M. tuberculosis infection has been disclosed (Felgner, P. et al, U.S. Pat. No. 7,927,818).

P. falciparum Proteome Microarray Chip Fabrication

4528 P. falciparum (Pf) genes, representing 50% of the Pf proteome, were selected and cloned into the Escherichia coli (E. coli) expression vector pXT7. Included among these genes were those corresponding to SEQ ID NOs:1-8. Custom polymerase chain reaction (PCR) primers comprising 20-bp gene-specific sequences with 33-bp adapter sequences were used to amplify target amplicons from Pf genomic DNA. The adapter sequences, which flank the target amplicons, are homologous to the adapter sequences at the ends of the linearized T7 expression vector pXT7 into which they were cloned. The homology allows the amplified PCR products to be cloned into the expression vector by in vivo homologous recombination in competent DH5a cells. The resulting clone mixtures were then verified by PCR using sequence specific primers and subsequently sequenced. For proteins microarray chip fabrication, Pf proteins were expressed in E. coli-based cell-free in vitro transcription and translation (IVTT) system (Rapid Translation System 100 High Yield [RTS 100 HY] kits from 5 PRIME, Gaithersburg, Md.) according to the manufacturer's instructions. Expressed Pf proteins were then printed onto nitrocellulose-coated glass FAST slides (Whatman, Piscataway, N.J.) using an OmniGrid Accent microarray printer (DigiLab, Piscataway, N.J.). Fabricated proteins microarray chips were QC using monoclonal anti-polyhistidine (clone His-1; Sigma-Aldrich, St. Louis, Mo.) and anti-hemagglutinin (clone 3F10; Roche, Indianapolis, Ind.) antibodies.

Serum Probing Using P. falciparum Protein Microarray

Sera from human trial subjects was collected no sooner than 7 days after administration of the last dose of the vaccine regimen, preferably within 10-14 days after administration of the last dose. Serum samples were diluted to 1:100 in Blocking Buffer containing 1 mg/mL E. coli lysate, and they were incubated at room temperature for 30 minutes with constant mixing. The arrays were rehydrated in Blocking Buffer for 30 minutes and probed with the pre-treated sera in duplicate overnight at 4° C. with constant agitation. The slides were then washed three times in TTBS and incubated in Cy3-labeled goat anti-human Ig (H+L; Jackson Immuno Research, West Grove, Pa.) diluted 1:400 in Blocking Buffer. The slides were then washed three times in TTBS and three times in TBS, followed by an ultrapure water wash. The slides were then air dried after brief centrifugation and analyzed using a Perkin Elmer ScanArray Express HT microarray scanner (Perkin Elmer, Waltham, Mass.). Intensities were quantified using QuantArray software.

Protein Microarray Data Analysis

Replicate data were averaged before normalizing the data. The data were calibrated and transformed using the variance stabilizing normalization (vsn) package in the R statistical environment (www.r-project.org). Differential reactivity analysis was then performed using a one-way regularized analysis of variance (ANOVA). p-Values were calculated using two-tailed Student's t-test of unequal variance. Seroreactive antigens with p-values less than 0.05 were considered differentially reactive and seroreactive antigens with p-values greater than 0.05 were considered cross-reactive. Finally, the data were retransformed into an approximate raw scale for bar plot visualizations prepared in GraphPad Prism. Receiver operating characteristic curves, respectively ROC analysis, were generated on Excel by adapting Support Vector Machines (SVMs), “e1071” and “ROCR” packages in R.

Surrogate Assay for Determining Protection

In the clinical trial of PfSPZ described by Seder et al., antibody titers to CSP were measured. Two weeks after the final PfSPF Vaccine administration there was a correlation between the total dosage of vaccine and results of PfCSP ELISA. However, as described herein, although when used alone, measurement of serum antibodies to CSP per se exhibits excellent sensitivity (100%) in identifying protected subjects, its specificity with regard to distinguishing between protected and unprotected subjects (77%) is too low to be useful as a clinical substitute for CHMI. As disclosed herein, the multiplex reactivity of serum antibodies from human subjects directed to Pf antigens of CSP and MSP5 demonstrate excellent sensitivity and specificity and are therefore diagnostic of immunologic protection against Pf-caused malaria. Using the summation of CSP and MSP5, assays demonstrate 90-92% sensitivity (preferably, about 92%) with 87-89% specificity (preferably, about 89%). When the results of MSP5 are multiplied by 100 (100×) and added to CSP, assays demonstrate 98-100% sensitivity (preferably, about 100%) with a specificity of 82-84% (preferably, about 84%).

In certain embodiments, assays have been developed in which immunological determinants of CSP and MSP5 are affixed on a chip as a microarray as described above. These chips are provided to the clinician as a surrogate assay for protective efficacy, for example as part of a clinical trial protocol, either in lieu of or in addition to CHMI. Additionally, these assays can be used to screen a population of subjects that have potentially been exposed to PfSPZ to determine those in need of vaccination. Probing of the sera of subjects is conducted and evaluated as described above. Alternatively, other immunodeterminative tests can be employed to determine sero-reactivity to specific, identified multiplex Pf immunological determinants. Sera can be assessed for antibodies against PfSPZ by immunofluorescence assay (IFA) (T. C. Luke, et al., (2003)) or recombinant to CSP and MSP5 or other Pf multiplex combinations by enzyme linked immunosorbent assay (ELISA) as described (T. C. Luke, et al., (2003)).

Assays of humoral antibodies of vaccinated individuals that are easy to administer and have substantial sensitivity and specificity for identifying those individuals that are or are not protected will have great value, not only during the clinical testing of malaria vaccines, but also as a public health tool during post licensure vaccination campaigns. In addition, assays of the application that can be processed quickly and provide rapid results provide an advantage over current assays (e.g., CHMI) for protective immunity.

In some embodiments, the methods of the application are used to determine a satisfactory end point for vaccination. For example, the methods of the application can be used to determine whether protective immunity has been achieved and, optionally, whether further treatment is needed.

In one embodiment, the end point has been achieved and no further vaccine is administered to the subject when antibodies that specifically immunoreact with at least two Pf antigens disclosed herein (e.g., CSP encoding nucleic acids (SEQ ID NO:1) and MSP5 encoding nucleic acids (SEQ ID NO:2) or antigenic fragments of the polypeptides encoded by nucleic acids SEQ ID NO:1 and SEQ ID NO:2) are detected in a sample from the subject.

In another embodiment, the end point is not achieved and a subject is given a further dose or inoculated with a P. falciparum vaccine when antibodies that specifically immunoreact with at least two Pf antigens disclosed herein (e.g., CSP encoding nucleic acids (SEQ ID NO:1) and MSP5 encoding nucleic acids (SEQ ID NO:2) or antigenic fragments of the polypeptides encoded by nucleic acids SEQ ID NO:1 and SEQ ID NO:2) are not detected.

### EXAMPLES

### Example 1

A clinical trial testing the immunogenicity and efficacy of aseptic, radiation-attenuated, purified, cryopreserved sporozoites used as the immunogen in a vaccine formulation (Sanaria® PfSPZ Vaccine, provided by Sanaria Inc.), and administered by intravenous injection, resulted in 13 individuals that were protected against controlled human malaria infection (CHMI) and 19 that were not protected (Table 1). In the group receiving the highest dosage (675,000 total PfSPZ), 6 out of 6 individuals were protected; and the group receiving the next lower dosage (540,000 total PfSPZ) had 6 out of 9 protected (Seder, et al. Science 2013).

TABLE 1
Protection efficacy

| | | Total | # | # | | Protec- |
|---|---|---|---|---|---|---|
| PfSPZ per | | dosage of | volun- | Pro- | Protec- | tion % |
| Dose | Doses | PfSPZ | teers | tected | tion % | Combined |
| 7500 | 4 | 30000 | 3 | 0 | 0% | 0% |
| (low) | 6 | 45000 | 3 | 0 | 0% | |
| 30000 | 4 | 120000 | 9 | 1 | 11% | 9% |
| (medium) | 6 | 180000 | 2 | 0 | 0% | |
| 135000 | 4 | 540000 | 9 | 6 | 66% | 80% |
| (high) | 5 | 675000 | 6 | 6 | 100% | |

The 5-dose (675000 SPZ) group in which all subjects were protected differed in two potentially important ways from other groups: a) these subjects received the highest overall dosage of SPZ; and, b) there was a 7-week interval between the fourth and fifth doses. Therefore factors that may have contributed to the incremental increase in protection may be the total dosage of PfSPZ, the numbers of doses, or the increased interval of administration. Nevertheless, this study provides the opportunity to screen sera from both protect and unprotected individuals to attempt to identify surrogates of protection.

### Example 2

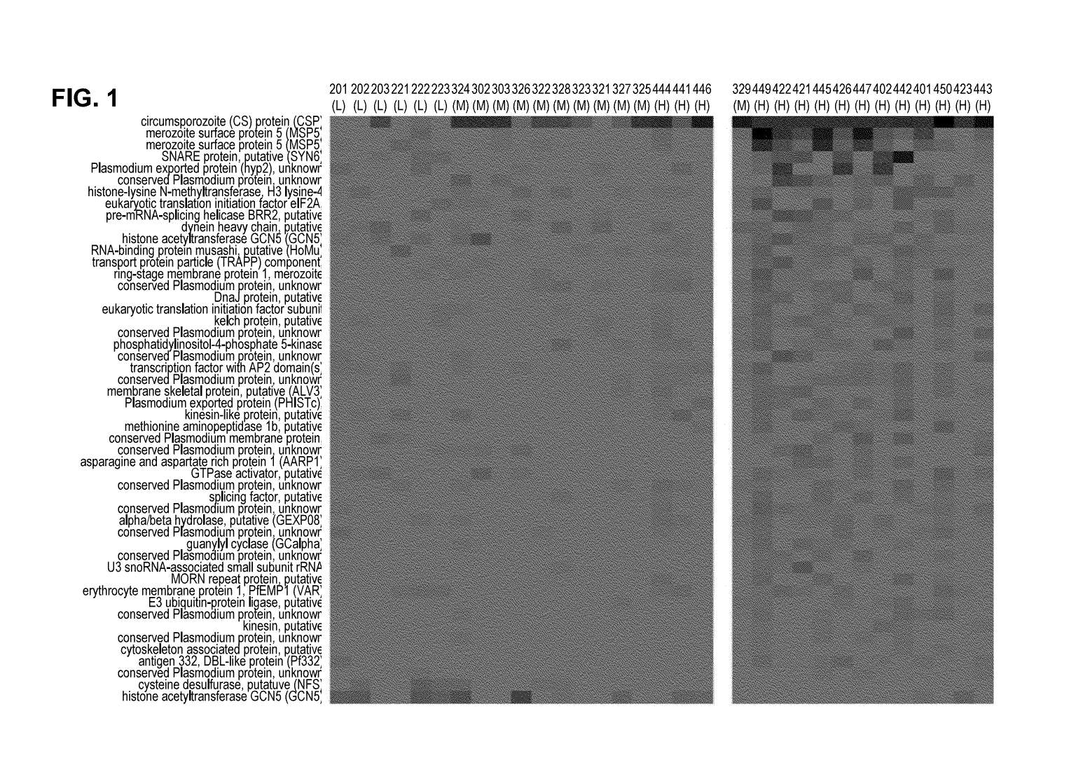

Serum from each of the subjects was drawn 14 days after administration of the last dose. Each serum sample was probed using the Pf microarray as described. As shown in the heat map of the top 50 differentially reactive serum antigens (FIG. 1), subjects receiving the high dosage (540,000 and 675,000) of Sanaria® PfSPZ Vaccine demonstrated the strongest reactivity. Two antigens (CSP and MSP5) proved to be the most reactive with regard to complementing serum antibodies.

### Example 3

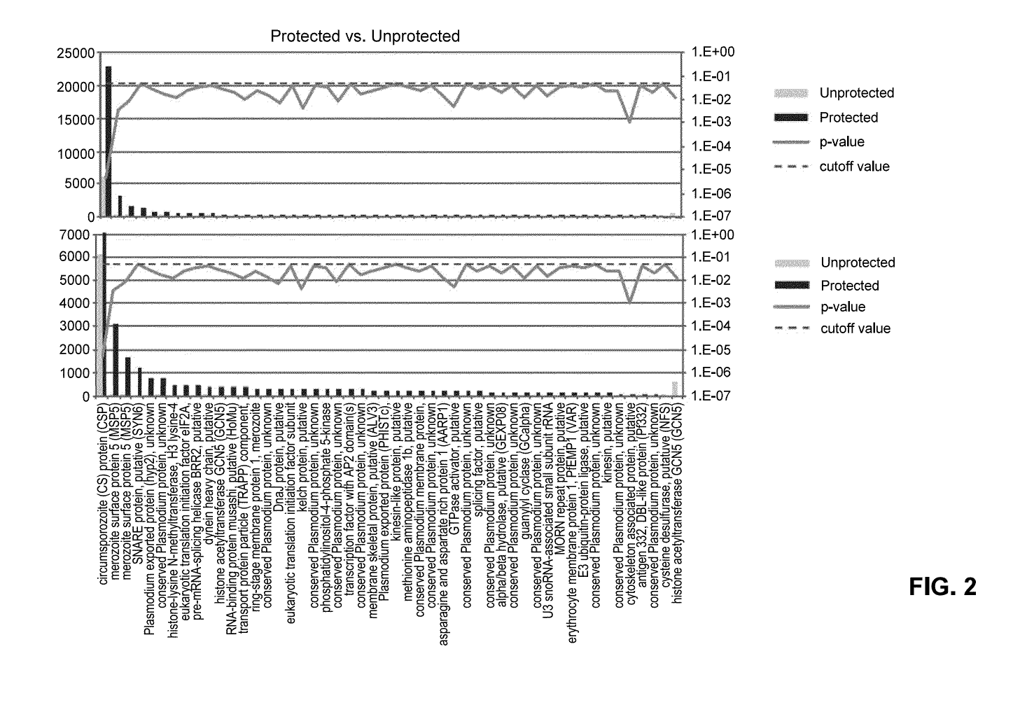

32 sera samples from the intravenous injection immunization trial of Sanaria® PfSPZ Vaccine, described in Example 1 above, were probed with Pf1000 microarray down-selected from a large array containing 4,528 Plasmodium falciparum (Pf) protein features representing 50% of the parasite proteome. Among the 32 sera samples, 13 were obtained from the protected individuals and 19 from the unprotected individuals. FIG. 2 shows the top 50 differentially reactive antigens comparing the protected and unprotected groups. The graph is sorted by the antigen reactivity of protected group along with the p-value and cut-off value.

### Example 4

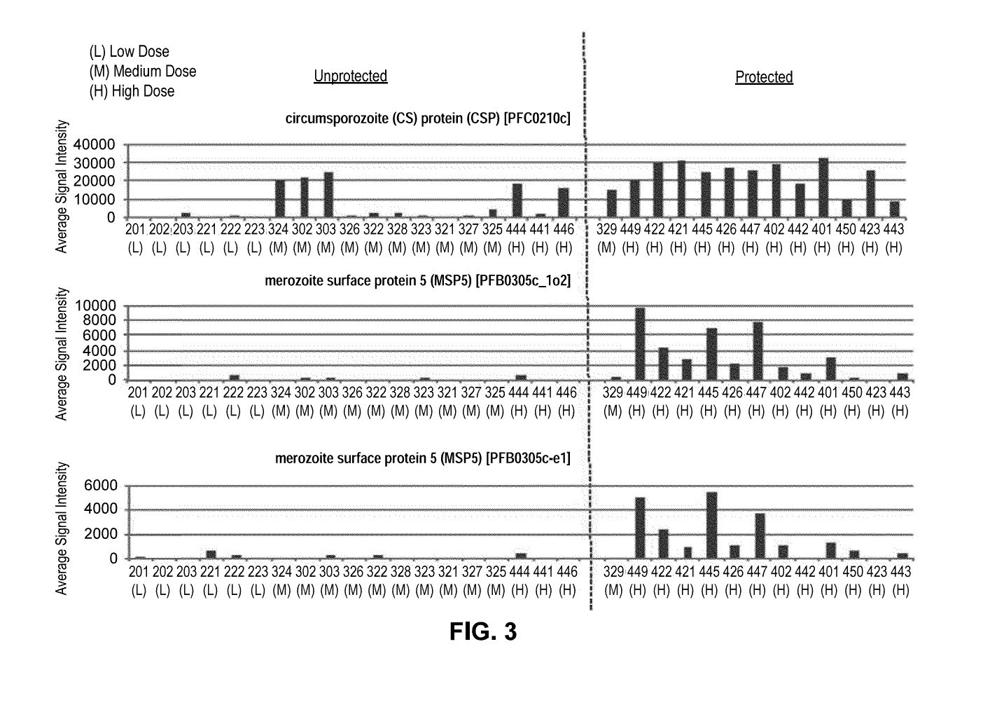

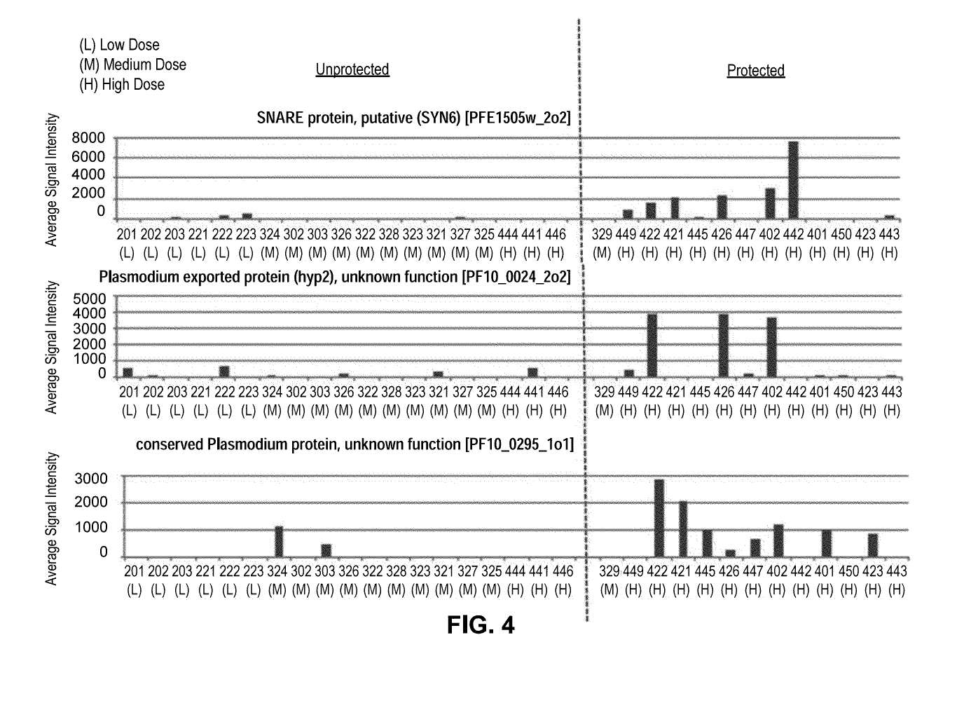

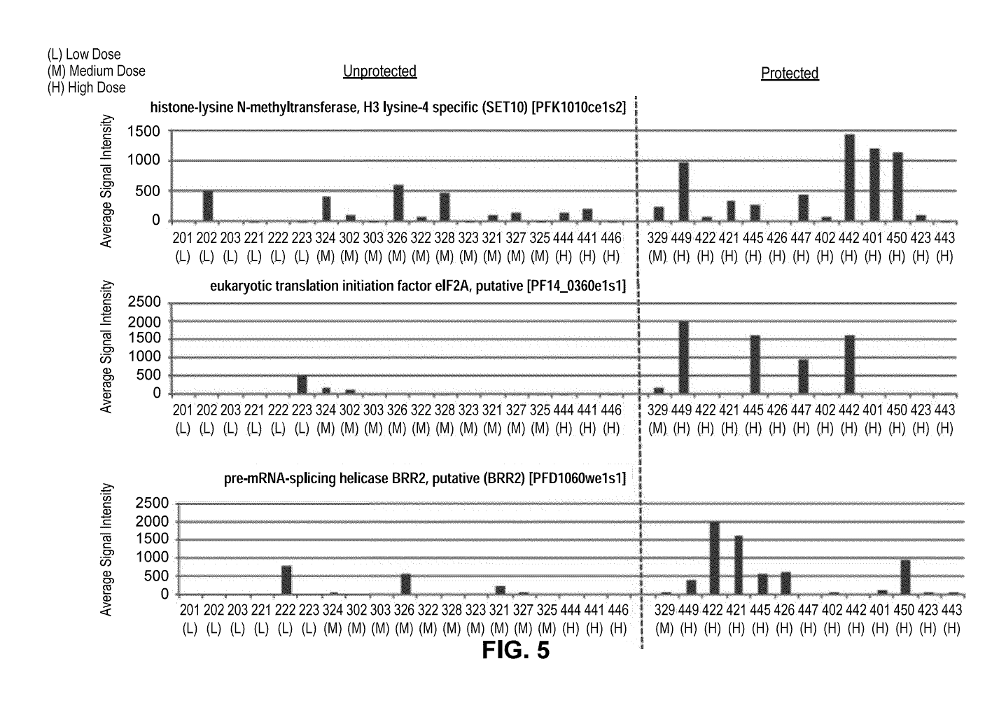

Analysis of individual sero-reactivity for 9 antigens from protected and unprotected subjects is shown. Six individuals are designated as low dose (L) (7500 SPZ/dose, administered 4-6 times; 11 as medium dose (M) (30,000 SPZ/dose, administered 4-6 times); and high dose (H) 135,000 SPZ/dose, administered 4-5 times). FIG. 3 shows reactivity to CSP (PFC0210c) and two segments of MSP5 (PFB0305c_1o2) and (PFB0305c_e1), the 3 antigens that demonstrate the strongest sensitivity of protected subjects. Antibodies to MSP5 demonstrates the highest selectivity between protected and unprotected subjects. FIG. 4 shows reactivity to SNARE proteins, putative (SYN6) (PFE1505w_2o2); Plasmodium exported protein (hyp2), unknown function (PF10_0024_2o2); and Conserved Plasmodium protein, unknown function (PF10_0295_1o1). FIG. 5 shows reactivity to Histone-lysine-N-methyltransferase, H3 lysine-4-specific (SET10) (PFL1010ce1s2); Eukaryotic translation initiation factor eIF2A, putative (PF14_0360e1s1); and Pre-mRNA-splicing helicase BRR2, putative (BRR2) (PFD1060we1s1).

### Example 5

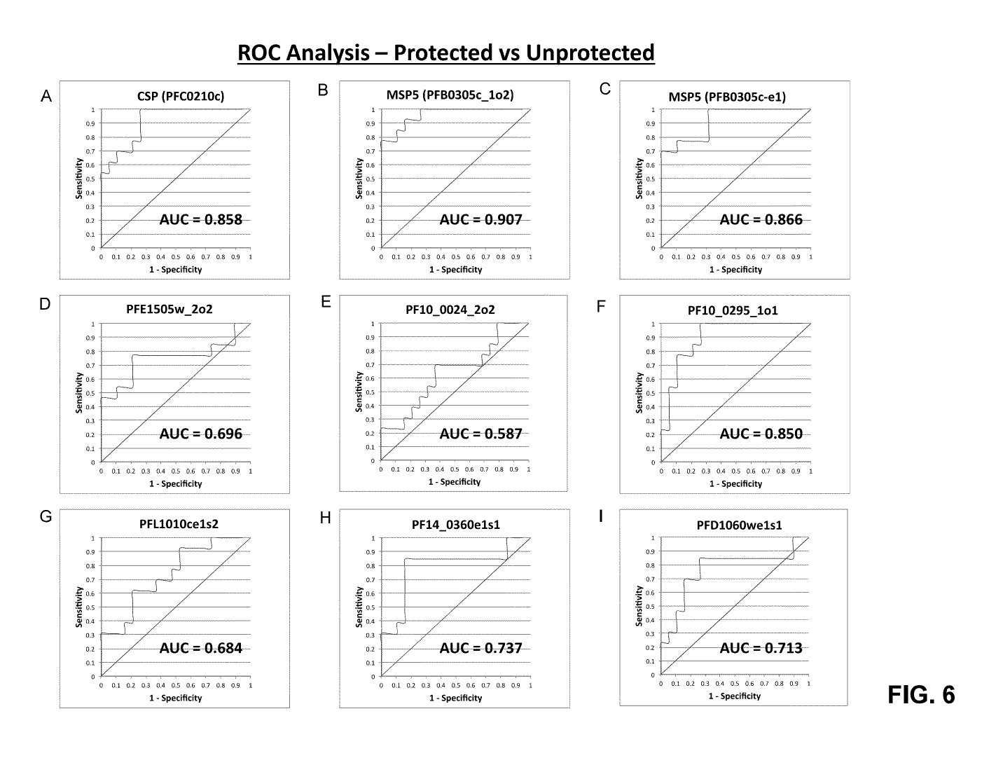

ROC statistical analysis graphs of protected versus unprotected groups in the top 9 sero-reactive antigens along with area under the curve (AUC) values is provided in FIG. 6. ROC was calculated by Excel with formula provided by the biostatistician, the results are similar to what obtained from SVM package e1071 under R environment. Results are summarized AUC analysis and listed in Table 2.

TABLE 2
AUC Rank of ROC Curves

| | Polynucleotide | | AUC |
|---|---|---|---|
| Gene ID | Sequence | AUC | Rank |
| circumsporozoite (CS) protein (CSP) | SEQ ID NO: 1 | 0.858 | 3 |
| [PFC0210c] | | | |
| merozoite surface protein 5 (MSP5) | SEQ ID NO: 2 | 0.907 | 1 |
| [PFB0305c_1o2] | | | |
| merozoite surface protein 5 (MSP5) | SEQ ID NO: 2 | 0.866 | 2 |
| [PFB0305c-e1] | | | |
| SNARE protein, putative (SYN6) | SEQ ID NO: 3 | 0.696 | 7 |
| (PFE1505w_2o2] | | | |
| Plasmodium exported protein (hyp2), | SEQ ID NO: 4 | 0.587 | 9 |
| unknown function [PF10_0024_2o2] | | | |
| conserved Plasmodium protein, unknown | SEQ ID NO: 5 | 0.850 | 4 |
| function [PF10_02951o1] | | | |
| histone-lysine N-methyltransferase, H3 | SEQ ID NO: 6 | 0.684 | 8 |
| lysine-4 specific (SET10) [PFL1010ce1s2] | | | |
| eukaryotic translation initiation factor | SEQ ID NO: 7 | 0.737 | 5 |
| eIF2A, putative [PF14_0360e1s1] | | | |
| pre-mRNA-splicing helicase BRR2, | SEQ ID NO: 8 | 0.713 | 6 |
| putative (BRR2) [PFD1060we1s1] | | | |

Antibodies against MSP5, CSP and conserved Plasmodium protein, unknown function [PF10_02951o1] were apparently higher in the protected group from ROC analysis. All had AUC values equal to or greater than 0.85. MSP5 was the top indicator of protection. CSP had the highest signal among all protected individuals, but also in some unprotected individual. Hence, a less specific indicator alone.

AUC of the top 9 antigens and their ranks were tabulated. Note: max AUC 1.0 indicates a perfect diagnosis antigen between the protected and unprotected groups.

Antibodies against MSP5, CSP and a conserved unknown Plasmodium protein (PF10_0295_1o1) are apparently higher in the protected group from ROC analysis. All have AUC values equal to or greater than 0.85.

MSP5 has highest AUC value, followed by CSP; and are good indicators for the protection.

As shown by ROC analysis, antibodies against MSP5, CSP and a conserved unknown Plasmodium protein (PF10_0295_1o1) are apparently the strongest indicators of the protected group. All 3 have AUC values equal to or greater than 0.85. Of these, MSP5 is the strongest indicator. On the other hand, CSP had the highest signal among all protected individuals, but also some unprotected individuals (i.e. less specific).

### Example 6

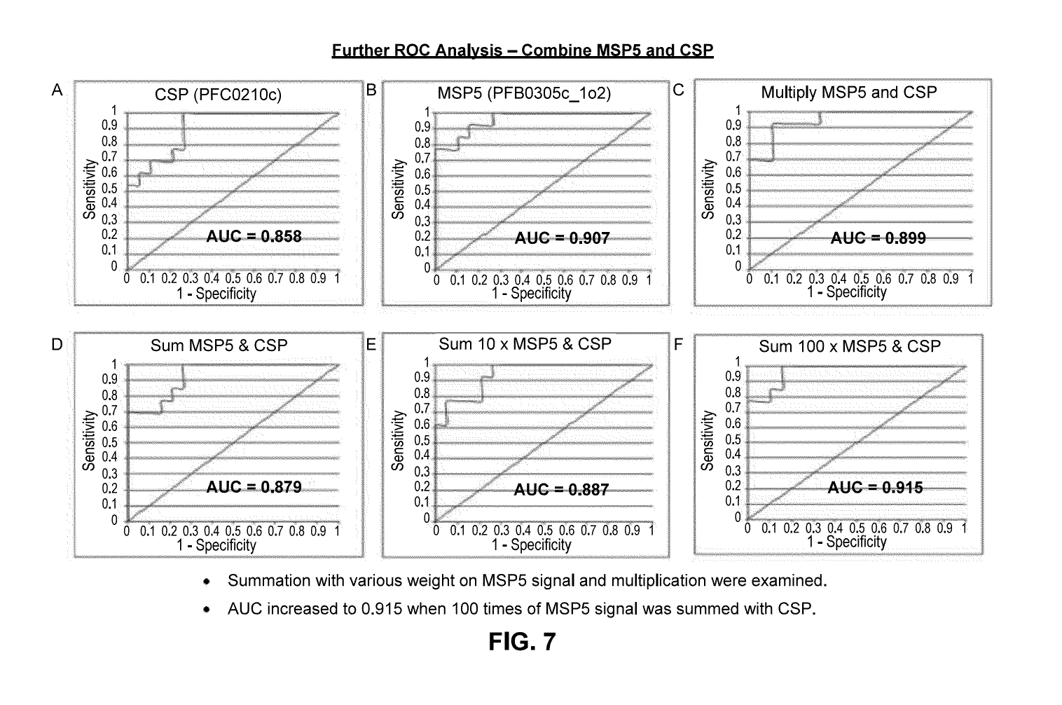

Further ROC analysis was performed by combining reactivity of MSP5 and CSP via various summation or multiplication to determine if AUC values can be improved. (FIG. 7). Highest AUC is obtained with the summation of 100×MSP5 and CSP. Note: Both MSP5, PFB0305c_1o2 and PFB0305c_e1, show similar reactivity profile and were determined to be two different clones of the same gene (SEQ ID NO:2). Use data from PFB0305c_1o2 for combined ROC analysis. The results are shown in Table 3.

TABLE 3
Further ROC Analysis - Combine MSP5 and CSP

| Data Treatment | AUC | Sensitivity | Specificity |
|---|---|---|---|
| Sum MSP5 & CSP | 0.879 | 100% | 74% |
| Sum 10xMSP5 & CSP | 0.887 | 100% | 74% |
| Sum 100x MSP5 & CSP | 0.915 | 100% | 84% |
| Multiply MSP5 & CSP | 0.899 | 92% | 89% |
| MSP5 | 0.907 | 77% | 100% |
| CSP | 0.858 | 100% | 74% |

Note:
MSP5 from PF80305c_1o2 was used for examination, since both MSP5 have similar signal profiles.
MSP5 alone shows 100% specificity, but only 77% sensitivity.
Contrary, CSP alone shows 100% sensitivity, but only 74% specificity.
Summation of 100x MSP5 signal and CSP signal maintains 100% sensitivity, and improves the specificity to 84% when compared to CSP alone.
Multiplication of MSP5 and CSP signal compromise the sensitivity to 92% and specificity to 89% of both antigens.

As shown MSP5 alone showed 100% specificity for protected subjects, but only 77% sensitivity. On the other hand, CSP alone showed 100% sensitivity but only 74% specificity. When the results for MSP5 is multiplied by 100 (100×) and added to the CSP signal, 100% sensitivity is maintained and the specificity improves to 84%. Alternatively, multiplication of the MSP5 and CSP signals drop the sensitivity to 92% and the specificity is 89%.

In the foregoing, the present invention has been described with reference to suitable embodiments, but these embodiments are only for purposes of understanding the invention and various alterations or modifications are possible so long as the present invention does not deviate from the claims that follow.

What is claimed is:

1. A method for determining a state of protective immunity against P. falciparum-induced malaria in a human subject said method comprising probing a human body fluid sample with Pf immunologic determinants comprising (i) a first Pf immunologic determinant having a sequence that is at least 80% identical to an amino acid sequence encoded by the nucleic acid sequence of SEQ ID NO: 1 or an antigenic fragment thereof; and (ii) a second Pf immunologic determinant having a sequence that is at least 80% identical to the amino acid sequence encoded by the nucleic acid sequence of SEQ ID NO:2 or an antigenic fragment thereof, and determining whether said human body fluid sample comprises antibodies that are specifically immunoreactive to said first and second Pf immunologic determinants, wherein specific immunoreactivity to said first and second Pf immunologic determinants indicates protective immunity in said subject.

2. The method of claim 1, further comprising probing said body fluid sample to a polypeptide having a sequence that is at least 80% identical to an amino acid sequence encoded by a nucleic acid sequence selected from the group consisting of SEQ ID NO:3, SEQ ID NO:4, SEQ ID NO:5, SEQ ID NO:6, SEQ ID NO:7, or SEQ ID NO:8, or antigenic fragments thereof, or a combination thereof; and determining whether said human body fluid sample comprises antibodies that are specifically immunoreactive to said polypeptide.

3. The method of claim 1, wherein the first Pf immunologic determinant has a sequence that is at least 85%, 90%, 95%, 99%, or 100% identical to an amino acid sequence encoded by the nucleic acid sequence of SEQ ID NO: 1 or an antigenic fragment thereof.

4. The method of claim 1, wherein the second Pf immunologic determinant has a sequence that is at least 85%, 90%, 95%, 99%, or 100% identical to an amino acid sequence encoded by the nucleic acid sequence of SEQ ID NO: 2 or an antigenic fragment thereof.

5. The method of claim 2, wherein the polypeptide has a sequence that is at least 85%, 90%, 95%, 99%, or 100% identical to an amino acid sequence encoded by the nucleic acid sequence selected from the group consisting of SEQ ID NO:3, SEQ ID NO:4, SEQ ID NO:5, SEQ ID NO:6, SEQ ID NO:7, or SEQ ID NO:8, or antigenic fragment thereof, or a combination thereof.

6. The method of claim 1, wherein said body fluid sample is serum.

7. The method of claim 1, where said antibody immunoreactivity is determined by enzyme-linked immunosorbent assay (ELISA).

8. The method of claim 1, wherein said first and second Pf immunologic determinants are provided as an array coupled to a solid phase.

9. The method of any one of claims 1-8, wherein said subject has first been inoculated with a P. falciparum vaccine.

10. The method of claim 9, wherein said vaccine is administered in multiple doses.

11. The method of claim 9, wherein said body fluid sample is collected no sooner than 7 days after the last dose of vaccine.

12. The method of claim 11, wherein said body fluid sample is collected 10-14 days after the last dose of vaccine.

13. The method of claim 1, where said immunoreactivity determination has a sensitivity of about 92% to 100%.

14. The method of claim 1 or 13, wherein said immunoreactivity determination has a specificity of at least about 89%.

15. The method of claim 1, where said immunoreactivity determination has a sensitivity of 100%.

16. The method of claim 1 or 15, where said immunoreactivity determination has a specificity of at least about 84%.

17. A subunit vaccine comprising (i) a polypeptide having a sequence that is at least 80% identical to the amino acid sequence encoded by a nucleic acid sequence comprising SEQ ID NO:1 or an antigenic fragment thereof and (ii) a polypeptide having a sequence that is at least 80% identical to the amino acid sequence encoded by a nucleic acid sequence comprising SEQ ID NO:2 or an antigenic fragment thereof.

18. The vaccine of claim 17, wherein the polypeptide of (i) has a sequence that is at least 85%, 90%, 95%, 99%, or 100% identical to an amino acid sequence encoded by the nucleic acid sequence of SEQ ID NO: 1 or an antigenic fragment thereof.

19. The vaccine of claim 17, wherein the polypeptide of (ii) has a sequence that is at least 85%, 90%, 95%, 99%, or 100% identical to an amino acid sequence encoded by the nucleic acid sequence of SEQ ID NO: 2 or an antigenic fragment thereof.

20. The vaccine of claim 17 further comprising (iii) a polypeptide having a sequence that is at least 80% identical to an amino acid sequence encoded by a nucleic acid sequence selected from the group consisting of SEQ ID NO:3, SEQ ID NO:4, SEQ ID NO:5, SEQ ID NO:6, SEQ ID NO:7, or SEQ ID NO:8, or antigenic fragments thereof, or a combination thereof.

21. The vaccine of claim 20, wherein the polypeptide of (iii) has a sequence that is at least 85%, 90%, 95%, 99%, or 100% identical to an amino acid sequence encoded by the nucleic acid sequence selected from the group consisting of SEQ ID NO:3, SEQ ID NO:4, SEQ ID NO:5, SEQ ID NO:6, SEQ ID NO:7, or SEQ ID NO:8, or antigenic fragments thereof, or a combination thereof.

22. A method for conferring protective against P. falciparum-caused malaria comprising administering the vaccine of any one of claims 17-21 to a human subject.

23. A method for identifying protective immunity against P. falciparum-induced malaria in a human subject, wherein the protective immunity is identified by the detection of antibodies specific to immunologic determinants of at least two Pf immunologic determinants coupled to a solid surface, wherein the method comprises:
(i) applying a body fluid sample from a subject to the solid surface, wherein the solid surface comprises (a) a polypeptide having a sequence that is at least 80%, 85%, 90%, 95%, 99%, or 100% identical to an amino acid sequence encoded by the nucleic acid sequence of SEQ ID NO: 1 or an antigenic fragment thereof and (b) a polypeptide having a sequence that is at least 80%, 85%, 90%, 95%, 99%, or 100% identical to an amino acid sequence encoded by the nucleic acid sequence of SEQ ID NO:2 or an antigenic fragment thereof;
(ii) applying a detection agent that binds antibody-Pf immunologic determinants to the solid surface of (i); and
(iii) identifying protective immunity against P. falciparum-induced malaria in said subject by detecting antibody binding to the polypeptide of (a) and the polypeptide of (b).

24. A method for identifying protective immunity against P. falciparum-induced malaria in a human subject, wherein the protective immunity is characterized by the presence of antibodies specific to at least two Pf immunologic determinants coupled to two or more solid surfaces, wherein the method comprises:
(i) applying a body fluid sample from a subject to a first solid surface comprising a polypeptide having a sequence that is at least 80%, 85%, 90%, 95%, 99%, or 100% identical to an amino acid sequence encoded by the nucleic acid sequence of SEQ ID NO: 1 or an antigenic fragment thereof;
(ii) applying the body fluid sample to a second solid surface comprising a polypeptide having a sequence that is at least 80%, 85%, 90%, 95%, 99%, or 100% identical to an amino acid sequence encoded by the nucleic acid sequence of SEQ ID NO:2 or an antigenic fragment thereof;
(iii) applying a detection agent to the first solid surface of (i) and the second solid surface of (ii); and
(iv) identifying protective immunity against P. falciparum-induced malaria in said subject by detecting antibody binding to the polypeptide of (i) and the polypeptide of (ii).

25. The method of claim 23, wherein the solid surface further comprises a polypeptide having a sequence that is at least 80%, 85%, 90%, 95%, 99%, or 100% identical to an amino acid sequence encoded by the nucleic acid sequence of SEQ ID NO:3, SEQ ID NO:4, SEQ ID NO:5, SEQ ID NO:6, SEQ ID NO:7, or SEQ ID NO:8, or antigenic fragments thereof, or a combination thereof.

26. The method of claim 24 further comprising applying the body fluid sample to a third solid surface comprising a polypeptide having a sequence that is at least 80%, 85%, 90%, 95%, 99%, or 100% identical to an amino acid sequence encoded by the nucleic acid sequence of SEQ ID NO:3, SEQ ID NO:4, SEQ ID NO:5, SEQ ID NO:6, SEQ ID NO:7, or SEQ ID NO:8, or antigenic fragments thereof, or a combination thereof.

27. A method for identifying protective immunity against P. falciparum-induced malaria in a human subject by a means for detecting the presence of antibodies present in a body fluid sample, that specifically bind to immunologic determinants of P. falciparum, said method comprising contacting the body fluid sample with (i) a polypeptide having a sequence that is at least 80%, 85%, 90%, 95%, 99%, or 100% identical to an amino acid sequence encoded by a polynucleotide comprising SEQ ID NO: 1 or an antigenic fragment thereof and (ii) a polypeptide having a sequence that is at least 80%, 85%, 90%, 95%, 99%, or 100% identical to an amino acid sequence encoded by a polynucleotide comprising SEQ ID NO:2 or an antigenic fragment thereof, and detecting antibody-polypeptide (i) and antibody-polypeptide (ii) complexes.

28. The method of claim 27, further comprising contacting the sample with one or more polypeptides having a sequence that is at least 80%, 85%, 90%, 95%, 99%, or 100% identical to an amino acid sequence encoded by the nucleic acid sequence of SEQ ID NO:3, SEQ ID NO:4, SEQ ID NO:5, SEQ ID NO:6, SEQ ID NO:7, or SEQ ID NO:8, or antigenic fragments thereof, or a combination thereof.

29. A method for identifying a human subject with protective immunity against P. falciparum-induced malaria comprising analyzing a body fluid sample of said subject for the presence or absence of antibodies that specifically immunoreact with (i) a polypeptide having a sequence that is at least 80%, 85%, 90%, 95%, 99%, or 100% identical to an amino acid sequence encoded by polynucleotides comprising SEQ ID NO:1 or an antigenic fragment thereof and (ii) a polypeptide having a sequence that is at least 80%, 85%, 90%, 95%, 99%, or 100% identical to an amino acid sequence encoded by polynucleotides comprising SEQ ID NO:2, or an antigenic fragment thereof, wherein the subject is identified as having protective immunity against P. falciparum-induced malaria if the antibodies that specifically immunoreact with the polypeptides of (i) and (ii) are detected.

30. A method for identifying a human subject lacking protective immunity against P. falciparum-induced malaria and conferring protective immunity to said subject comprising: (a) analyzing a body fluid sample of a subject for the presence or absence of antibodies that specifically immunoreact with: (i) a polypeptide having a sequence that is at least 80%, 85%, 90%, 95%, 99%, or 100% identical to an amino acid sequence encoded by polynucleotides comprising SEQ ID NO: 1 or an antigenic fragment thereof and (ii) a polypeptide having a sequence that is at least 80%, 85%, 90%, 95%, 99%, or 100% identical to an amino acid sequence encoded by polynucleotides comprising SEQ ID NO:2 or an antigenic fragment thereof, and (b) inoculating subjects having samples lacking antibodies that specifically immunoreact with the polypeptides of (i) and (ii) with a P. falciparum vaccine.

31. A method for conferring protective immunity against P. falciparum-induced malaria to a human subject in need of said protective immunity comprising: (a) requesting a test providing the analysis of a body fluid sample of a subject for detection of antibodies that specifically immunoreact with: (i) a polypeptide having a sequence that is at least 80%, 85%, 90%, 95%, 99%, or 100% identical to an amino acid sequence encoded by polynucleotides comprising SEQ ID NO: 1 or an antigenic fragment thereof and (ii) a polypeptide having a sequence that is at least 80%, 85%, 90%, 95%, 99%, or 100% identical to an amino acid sequence encoded by polynucleotides comprising SEQ ID NO:2 or an antigenic fragment thereof, and (b) inoculating subjects having samples lacking antibodies that specifically immunoreact with the polypeptides of (i) and (ii) with a P. falciparum vaccine.

32. A method for identifying a human subject lacking protective immunity against P. falciparum-induced malaria after being inoculated with a P. falciparum vaccine comprising: (a) inoculating a subject with a P. falciparum vaccine and (b) subsequently analyzing a body fluid sample of the subject for antibodies that specifically immunoreact with (i) a polypeptide having a sequence that is at least 80%, 85%, 90%, 95%, 99%, or 100% identical to an amino acid sequence encoded by polynucleotides comprising SEQ ID NO: 1 or an antigenic fragment thereof and (ii) a polypeptide having a sequence that is at least 80%, 85%, 90%, 95%, 99%, or 100% identical to an amino acid sequence encoded by polynucleotides comprising SEQ ID NO:2, or an antigenic fragment thereof, wherein when said body fluid samples lack antibodies that specifically immunoreact with the polypeptides of (i) and (ii) said subjects are identified as lacking protective immunity against P. falciparum-induced malaria.

33. A method for identifying a human subject lacking protective immunity against P. falciparum-induced malaria after being inoculated with a P. falciparum vaccine and providing protective immunity comprising: (a) inoculating a subject with a P. falciparum vaccine, (b) subsequently requesting a test providing the analysis of a body fluid sample of the subject for detection of antibodies that specifically immunoreact with two or more Pf immunologic determinants, wherein two of the Pf immunologic determinants are polypeptides having a sequence that is at least 80%, 85%, 90%, 95%, 99%, or 100% identical to an amino acid sequence encoded by polynucleotides comprising SEQ ID NO: 1 or an antigenic fragment thereof; and SEQ ID NO:2 or an antigenic fragment thereof, and (c) inoculating subjects having a sample lacking antibodies that specifically immunoreact with the polypeptides of (i) and (ii) with an additional dose of the P. falciparum vaccine.

34. The method of any one of claims 30-33, wherein the subject's sample further lacks antibodies that specifically immunoreact with one or more of the polypeptides having a sequence that is at least 80%, 85%, 90%, 95%, 99%, or 100% identical to an amino acid sequence encoded by the nucleic acid sequence of SEQ ID NO:3, SEQ ID NO:4, SEQ ID NO:5, SEQ ID NO:6, SEQ ID NO:7, or SEQ ID NO:8, or antigenic fragments thereof, or a combination thereof.

35. A method for vaccinating a human subject at risk for P. falciparum-induced malaria, wherein the method comprises:
(i) determining immunoreactivity of antibodies in a body fluid sample from said subject to at least two recombinant Pf immunologic determinants, wherein two of the recombinant Pf immunologic determinants are (a) a polypeptide having a sequence that is at least 80%, 85%, 90%, 95%, 99%, or 100% identical to an amino acid sequence encoded by a polynucleotide comprising SEQ ID NO: 1 or an antigenic fragment thereof and (b) a polypeptide having a sequence that is at least 80%, 85%, 90%, 95%, 99%, or 100% identical to an amino acid sequence encoded by a polynucleotide comprising SEQ ID NO:2 or an antigenic fragment thereof;
(ii) identifying subjects with negative immunoreactivity to at least the two recombinant PF immunologic determinants of (i); and
(iii) administering a P. falciparum vaccine to the subjects identified in (ii).

36. The method of claims 35, wherein said method has a specificity of at least about 84%.

37. The method of claim 35, where said method has a specificity of at least about 89%.

38. A kit for identifying a human subject lacking protective immunity against P. falciparum-induced malaria comprising, in one or more containers, (a) a recombinant polypeptide having a sequence that is at least 80%, 85%, 90%, 95%, 99%, or 100% identical to an amino acid sequence encoded by a polynucleotide comprising SEQ ID NO: 1 or an antigenic fragment thereof; (b) a recombinant polypeptide having a sequence that is at least 80%, 85%, 90%, 95%, 99%, or 100% identical to an amino acid sequence encoded by a polynucleotide comprising SEQ ID NO:2 or an antigenic fragment thereof, wherein (a) and (b) are immobilized on one or more solid supports; and (c) an immunologic determinants-antibody detection reagent.


SEQUENCE LISTING (8 sequences)

SEQ ID NO: 1 - DNA, 1194 bp, Plasmodium falciparum
  misc_feature (1)..(1194): PFC0210c
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattag

SEQ ID NO: 2 - DNA, 819 bp, Plasmodium falciparum
  misc_feature (1)..(819): PFB0305c
atgaatatattatgtattctatcatatatttattttttcgttattttttatagtttgaatttaaataataaaaatgaaaattttttggttgtcagaagattaatgaatgacgaaaaaggagaaggtggttttacaagtaaaaataaagagaatggaaataataatagaaataatgaaaatgaactaaaagaagaaggttctttacctactaagatgaatgaaaaaaattccaattcatcagataaacagccaaatgatatttcacatgatgaatcaaagagcaattctaataattcacaaaatatccaaaaagaacctgaagaaaaagagaacagtaaccctaatttagatagtagtgaaaattcgagtgaaagcgcaacacgttctgttgatatatcagaacataattctaataatccagagacgaaagaagagaatggagaagaacctttagatcttgaaattaatgagaatgcagaaataggtcaagaacctccaaatagattacattttgacaatgtagatgatgaggtgccacattatagcgccctaagatataataaagtagaaaaaaatgtaaccgatgaaatgttattatataatatgatgagtgatcaaaatagaaaatcatgtgccataaataatggtggatgttctgatgatcaaatatgtataaatataaataatataggagttaaatgtatatgtaaggatggatatttacttggtacgaaatgtataatattgaattcttattcttgccatccatttttttctattcttatttatattacattgtttttgttattattcgtttaa

SEQ ID NO: 3 - DNA, 678 bp, Plasmodium falciparum
  misc_feature (1)..(678): PFE1505w
atggatttatgggacaaagattatcagaaagctttacaaacgggaaaagatataaagaaattattaaagcaaagtgagaaagaaaaaaagaaagctaaaaatagagctatattgagaggtaaaataacagaatttaatcagagtgtaaaatttcttgtacaccaattaagtaatgattatataaagaatgataagcattttataaaaaatgaaacgaaatatatgaataaagtgtcagcattagaaaaggtgaaaaaggaaataggtaccttatatgaagattatgcatcaacaaatgaaggggagatttctttaaatatgaatatggaacttttaaacgaattcgaaaatgaagagaatacatatttgaacgatttgaataaagaagagctgttattaaaacaaaacaaattaatgaaattacaagatgaacaattaaatttccttgaaggtacaacccataatttaaaaaatattagttataatataaatagtgaaatacaagtacataatgaattattagatgatattgacagagatatggacgaaaccaacaatttattagatagaaatagaaatatttttgaaagggttactagtaatacaagtaattattttttatattttttaattgttattttaacagcaagtcttatattctttataatgatactctaa

SEQ ID NO: 4 - DNA, 1410 bp, Plasmodium falciparum
  misc_feature (1)..(1410): PF10_0024
atgtttatatgtatatattttttttataataatatagtaattgaatctacacctataaatgtcataataaaaaaaaaaaaaaaaatgactaatttaagatatggaaatacatttaattttatatgtaataaatatgatgaagttatacaacacggttgtattaatttagataatatgcataaaaataataaaaagggaaataagaaaaaatatattaatatttttccaattatgaaagttttagtaatttgtctcttattgtcattagtgaataaatcaaatgataatacatataatgaggatatatttcaaaaggataaagagaattgtggtggtctaagatacaaaagattattagcagaaccaattttcggatttgatgtcatgtatgatttatttaaagataattttttagaacaaattggttataatgatgcacaaaaaaatgaattaaaagtttctatgaaatcgttttttcaaagttcaaatagtcaagaatatacctattctagacgagaagataatattcatagtattagtaaatcaaatgtgacaaacgtgaaaagtgtgaaatttgaagaaccacaagatgagaaaaatttaattacaattgaagaaataaaagcagacaataatgattcaaaagaaaaaaaaaatgaaataactaatataaacgtggatgaaaaaaaggatttggaaagtgtaatttccagtgaagaagaaaaaacaccaactagtaataatataaggaaaaagagaaagaccaaaaggtctcataagaataaagacagtaacacatcttatgataatatagaattgaataaaatattaaaagatgataatatagaagtaaataaaacattaaaagaagataatatagaagtaaataatacattagaagaagataatatagaagtgaataatacattaaaagaagataatatagaagttgaaaaactaattgatgaaaacagagagttaattaaaaaagaatctatatataaaaataaattaaaagaagataatatagaagttgaaaaactaattgatgaaaacagagagttaattaaaaaagaatctatatataaaaataaattaaaagaagataatatagaagttgaagatctaattgatgaaaacagaaagttaattaaaaaagaatctatatataaaaataaattagaagaagaacctgttgaaaaaggaataaaaaggaaaaattatctttttcttatacttgaaaaggcaaaagaaattcccccaggtgttattgtaggattattttctattttttacgttgcactaggagaaaatagatcatctttaattttattaattgtaatatttattttaacagtacatacatatttggaagtaaaaagaatatgtcgattcttaaatatagattaa

SEQ ID NO: 5 - DNA, 1353 bp, Plasmodium falciparum
  misc_feature (1)..(1353): PF10_0295
atggcgagagtgcggaaggggaaagccatgcccgtaatgtctaaaaactcaatattcgttttaaatataattatgactataggttttataagtgctatacctcagattttcacaactttaatgacatcatggagagaagctgaacctatagaatataaatacatgtttagaggaagtgaacattccttatatgttgattatacatattacggtttatataaagtagtatatgataataaacatgtagagacttggacacaacgagtacagaatatgaaaaggaaaggtatagaaggtatacaacaaggtaaaaatagtgaatcagctggtagcttatcattatggacatctgtatgtcctgaagcttgtagagatgctattgttagacgtatagaagcatatgaaagagtttcttttatatctttagtacttttatgtggtattgtcatatcatgtactattgttatcttgtcagttggttggaatttattattttcaaaaagcatatttatattaatgggatgttttattttttcatttgttataaatgctggtatcggtacctattggtattatgaaacagatatgtcgtggaactctataactaaagcacaacaatatcctttcccccggtgctcacattgtttttacgtttttatgattactacaggaatatatgcactctgttttcttagcttattattattagacttatttaataaaggcaaacaaaaatcatcacaccgagatcaacttaatgcacataatagaaaccctatgaataataaagctatgtaccaacctatgttcgataatcaaccgggtatgatgatgcagagaagtgctagttactctaatattatgccatttgttaaaggaatgaataataatgattattctaattatatgcaatataataaaatgggaatgaatatgaacatgaatatgaaccaaatgccacaacaaggattcccaaattttagaaatatgggatccaatgtgggcccaaatatgggatcacctatgggatcacctatgggaccatctatgggatcacctatgggtccatctatgggatcacccatgggaccaaatatggggtcccctatgagttctccaatgggttcacatatgggtcccaatttgaatcccaattttttccctcaacaatcaaggcaatattcttattcagtttccccaacgtatcaacaaaacatgcctaattttaataacttttcaaatagacatatgccatctatgtctgatttatattttgcaagacaatattcagggatgaaatttggggacatgaataacagtccatttgattcacagaaaccatataaattttaa

SEQ ID NO: 6 - DNA, 6990 bp, Plasmodium falciparum
  misc_feature (1)..(6990): PFL1010c
atgagtatatctccattgaaatatgaatccaaaatattgagcaacctagctaatatgctaaaattgagcgaaaagtcagatgatgaaatattaagtgaaataaatcaaggtaatgaaaataaagtttttgaaattgatgatgatgttaaacaagtggttgatttgaataaaattaattgcacatgtgaagaatcaaagaaaaaaatagaagagtgtgataaaaagaaatttgtatataattataatattgaaacatatggagtagatttttttcttgatatttgtaatgctttgtcaagatctaattataaaccttatttaagtgaagaagggaaaaaagaaataatgaatggattaagagtaagaagaaattcaaatttatataaatatgtttctttttttttaagtaaagaacgaatgaataaagaaagaatggatgacggcaaaaaggtagaaaatatttatcattgttttaaatgtttaaaaagtgtattacatatatgtattgatgatgaagatagtaataataataataataataataataagatgaaaactgcattgtatttaaaaaataataatagtataactgaacaaatcgaaaataaaattgagaatgatatattaaatgaacaaaacagtagtcttgaaaaagtaaatcatagaaaaggagttatatctgtttcaaaatataaatcagaattaacttctattactagaatgtctaatgaaataataatgcaaactatattagaacataccaaggatatgggtataaaaaaggatacaaccaaaaatatacaatcaagaaatgataataaaaatgatcatggtgatgaagatgatcatagaaccaataacatcaatagtaacatgaagaatagtaataataataataataataataataataatattataaaaaaaagcaaaacttatgaaagcgtcaaagtaagaaataattattattataatgatgatcatgattatgataatcatatttatcaaactgaaaatgttatagataataataataccgaacccacaaaaagaaatattccgaaaagaacagttgtacagaaaaaatcctatgtacagcgaaaaacaaacataacaaataaaaagagaatttctcaaaataaaccggtaacaaggaattcatataaaaaaaatgatagaataaatactacatgtgatgtaagagcaagcaaaattaaggatactatatatgaaaagaaaaagaagaaggtcagaaggagttttgaagtaaatgcgaatcgaagaaaaaggggtgcaggacaaaagggcgtattttctgatgaagaaggaatgaaaatggataacgataataaaattgatgataacacattaaacaataacaaaattgatggtaatatattagacaataacaaaattgatggtaatatattagacaataacaaaattgatggtaatatattagacaataacaaaattgatggtaatatatcagacaataacaaaattgatgataaaacattaaacaataacaaaattgatgataaaacatcagacaataacaaaattgatgatatcatatcagaccataacaaaattgatggtaatacaccaaacaataacaaaattgatgataacacatcaaacaataacaaaattgatggtaatacaccaaacaataacaaaattgatgataacactttgaaagataatataactaataacatattggatgataatgctgtaccaaaggaggtagataataattgtgaaaatgataaaaatgtacataataaagaaagctgtagaaatgatatatattcatccatacagataagtaatacgtataataatacaatgatcaattctacatttgtagaagaaaaaaatataataagtaatattatggacaatacaaatatatatccatataatttatgcaatgtaaataatatggaagatcctaatttgagtaataacccagaaaaagaatatattaaagaaaatcatttgtgtgatttaccaaatgatattataacttctaatggtgaagatataaaaaataaggagatttataaaatggatcatcccaataaagctttactatcaaataatacaaatgagaaagattatgattataacgaaagtgataacgcatcttattctacagatgattctaaatatgtaaaatatttaaagaagaaaaagaaattacaagaacaaggtaaaattattatagatctaaatttatttaatggaaattctgtgaaaacaacaaataaacgtgataaagaagaaaagttacaagaaaaattaagaatgaaacaaataaagaagcttttagatgaaaaacaaatggttatatgtaaaattgaagatatagctaaaaagagaaataaaaaatatgtaaaagcacaagtattatctaaagattctaaaattgaacgagcctattttagtcataaaatgaaaaaattttataattccaaatctagctattttggttgtggatggtcaagtaacaaaaaggaatggactccatttattcatgcacctttctttgaaaaccaacataacgcaatttataaaaatagaaataaaaaattgtatgaagaaatatatgatacattattacatggaagaatacatcctgacattaaagttgttgaattaaaagatcataaacatcctattaggttgtgtactccgtataatgaagattgttattctgttgtatatacaggtaaaaaaataaatgcaactgatgatagagttatatttggtgagtacacaggatatgtagctaataataaagaattgtcacaagaaaaacatcaatatatgtttgctttaacattcaataagaaagtatttaatgataggaaacatgtcgtttttattaatgaagttgagttggatgaagagcaaaattattctagtcatgttgtatcaaatgtgatgaataaagttaaggctcatgaaaaaaataatataaataatacaaacatttataatagtccgcacattttgaatggtaataataataataatataaatagtaatactacagaggaaactatgtcaaatagaaagattgataagattaagcccgtttcgaatgtaattatcccagattataataaccttaatagttatattaataagtgtaaaacaaatgaacataataaaaagtcaagagatataaataatttaataatacttcctgataattatacatatgctgtagattcatcttatatgtttaatgaaatgtctctagtaaatcattataaaacatgtagtgtatttaacaactttgatttcagaataaattctgaatggcaattagtatacttggatggatggccacatattattctaacatcaattccaggtgttgaaattaatcccggtgaggaaatttttgctgattttggttttgaatggtttgaaaaagttaatgatatatgtttaaatgaatttattaaaaataactatttttatagattatataaattaaatacatcgagagaaattcttttcaatggaatggaggatattgtggaaaaatataatttattaaaaaatcatattacttgtaatatatgtatgcataatgttaatactgatggtaataatttcattttatgttcaggatgtaatcatgtttatcatttaaagtgtgtacataaatttaacaccgaagttaatgaaaattatgaatggttctgttctagttgtcttcaattttgttttaatattataaaacaaaaggagtttgtagattatatagaaaaggaaaaccaaaagaggttcattcagcttttagatgatttaaatggggataatttggatactgtcaataatgaaaagaaatctgatgctctggataaattaaataattataattgtgtgggtaatataaataattctaatagtattgataatatagataatagggatggtctagataaccctaataactcaaatatctcttatggcgttaaaaactcaaacatttcttatgatgctaataattcaaatatttcatatgatgctaataattcaaacatttcttatggagttaataattcaaacatttcttatggcgctgataattcaaacatttctcatagctttaataattcaaacatttctcatagctttaataattcaaacatttctcatagcgctaataattcaaacatttctcatagcgctaataactctaacagcgaagataaaactaataattgtaacatcgaagataaaactaataattgtaacatcgaagataaaactaataattgtaacagcgaagataaaactaataattgtaacaacgaagataaaactaataattgtaacatcgaagataaaactaataattgtaacaatttagataaggaagatgacataaacgatatgaacaaaataaacaaatcaaaccaaattataattaaagatgtcaatgataataatttatttcctgttgaaaataatgataagcagaaatatttggaagttttagaaaatataagaaaacttctacagtgtaaagaaaatatcgatgatttatttaaaaataaagaatatgtaaatagctttttgcaaaaaatagatgatgaaatgaatatgtttgaaaataaaagaagcgaagatgaatctgttaaacttgatgatttaaaacatatttttgaaaatagttttgaacttcatttattaatagattataagaaaaaattagaagatttattggaaagtacagaaaaggtggattcatttttgaatgataagaatgttttgaatactatgaaaataaggaaaagaacattatcttttttgatggaaaacaaaaagtatatagataattttatacaaatgaatgaagaaaaagattacgcaaaaattaatgagtgtgaaaagaagaaagaagaagatattattcataatacaacatgtagaaaagtagaaaaactagaagataggattattcaaaatagccataataatcaattagcaaatcctgaatattgtagtacattaaaaggagaagaggataaatccgtaagtttgatagataataaatcaatgtgtgaaagggtagataatcatagtatttgtaatgaagtaaattcaagcatatcaagtacgtacgaaaaaacattgtacaataaatctaataatataattagaaatttaaaggaaatgaaaaagtatgaaagagaatgccaaaataataacagttatagtaatatgtattgtagctatatgatgaaattaagtaagaaaatattaggatttcctttaataaaagacttttctaaaggcttaagtacacttgaaccatctttacctttaaatgatcatttaaaaaaattatcggtttgttcaaattgttatagtaaacatcatgatttagcaaaagcaattatttgtagagtgacaaaaatgcattttgaagctaattataatgattacttaactgacgaagatttatttaaaacatccagtgaatttatccaatcagtcataagagaattagctaatactgtaaaagagtacaggaaaaaggaattgaatagggtatatttgcaacatgcaacagaaaatgatagtatgatactagatagagttaataaggatatgagcttggaattaaattataataatgttacaccaactagaactagtattcacaattgtagttgtacaaaaacgaataaggaatataatgaatccttttataaaattgtgagtccttcatttaataatgacactgttgaaaagagtaatattatcatacaagaaaataaccatattcgaaataatgaaattttacaattaggtgaagaagaagtgggaaattacgaatgtcagaataaagaagttattggttctgatacaaaagagcatacaaatgaaaggataaacgatcaaacaagtgaggaaaaatgtcatgatgatttctattcaaaggatatgcatatattatataaaaatacctttttaccaagagaagaaatagataactgtagtatagaaaaaattaatgatgttgaagagaaaaaaatgacacaccaacatgaagaaaatgacatattaaaggatgtaataaatgatacattaaatgatatgtcaaatgatgttccaaatgatatgtcaaatgatgttccaaatggtatgtcaaatgatgttccaaatggtatgtcaaatgatgttccaaatgatatatcaaatgatgttccaaatgatatatcaaatgatgtgtcaaatgatgttccaaatgatgtgtcaaatgatatatcaaatgatgtgtcaaataatgttccaaatgatgtgtcaaatgatgtgccaaatgatgtgccaaatgatgtgccaaatgatgtgccaaatgatgttcctaatgatacatcgatcactaatttgtatgaacaaaaaaatgatttgaacagctatgagaaatcttgtaaattaaataattttattccccttataggagtagaattaggaaagacgaaattccaaagagaatttacaaatggtacttttgttggtagagttacagaacaaataaaggacgaaaataataataatttttttgttgtaacttatgaagatggagatgttgaatggataactccatcctttttatttcaagaaattctaaaacaatctacaaataatactacatatcctttagctagtacttttaaagatatattttatgaagattttaaaaaggatataaaactaaacaaccattcttatgaattaaaaattgaaaagaaaaagcgaaaaagtatgtttgaatatgttcctaataataatgtaaccaaaagacaaaggcatgcattcgaagaaaatgtattaaaaaaaaaaaatacccatcaggaagttaatacatcacaaagaagtttgagtaaattgaaaaatgtagtatttaatgattcacttaaaaaaacaagaaaaagaactatgtctacaagttga

SEQ ID NO: 7 - DNA, 1986 bp, Plasmodium falciparum
  misc_feature (1)..(1986): PF14_0360
atgataccagataataatgaaaacaataataatttatatttcttagcatttagtaaaaataaagtgacgatttatgaatatgatgtggatttaagtaaaaatattgtagatgatattaatgatgaaattaaaagatgtgaagaggaagatgagaaaaataaaataaatgaaaatacagttcctatgacgaatgatgaaaaaaaagttgatatgaatcatataagaaataataatataataaataataatacaaacaatacgaatcagataaaaaaatctacaaataaatttaataatgattttgttttaaatcgtggtattaaaatatataaggaatatgacgatgttgaactagccacatgtacaaatgactataaaaaaattatccttgtaagaaaaagtgatttaaatgtagccgaaattattgatataaaaagtgaaaataaaaatgtgacatgtataaaaacaaaaacacaaattaaaaggattattacgagtcctagggataactacattgttttacattgtcaatacaagcctgatataacaaatacgaatttgtatgtatataaattaaatgtaaaatcttctaagaaaaaaaaaaagaaagacaaaaagaattcagaatgtgaaaatataaagggtgaagaaaattcagtaggaaaagaaaatgataattatgaagaagaaaagaaaaatattaatatgaataataataatgtgccgatacataataataataataacagtgataataacgaatcaactaatgataatattatatataatgaaaaccttattatagaacttacactcaaaagttatagtaacaaaaattggcctttcttcaaatggagtgatagcgaatctatatgcacttgtctaattaataaccaagtatatatttataaagataataatttcaatacaactgtgaacaaactgaaattagataatattgaattttttgatatatctcctgaattaaataataagaaaggaatattaatagctacatatgaacaaggaagtaaaggaaatccatcaatttttaaaatatttcatattgataatttaaataaacacatatactcaaaaaacttttttaattcagatgaaataaaactcaagtggaataaaaatgggagttccttattattacaaatacatacccaagtagataaagagaaacagtcatattatggttcaagcaatttatattttattgatactgtaacattaaaggatgtaaatgttatgactaataaaggattaatatatgataccatatggtcaaataatcaaaataaattttatgtatgtaaaggtgaaatcccagcagaaattgttctacatgataaaaatggaaatgtctcgcattcatatggaagacataaatttaatacgctcaaattaaattataatgaaaaattattattaacaggaggatttggaaacttaagtggagatatatctatttggaatacctcaaccaaaaaagaaattacaaaaaccaaatcatcatgtgcagtcatatgtgaattttttaatgatgggaaacatttcttaacagctacaactcatccaagattaagagttgataataatttgaaaatatttaaatataatggtttaatagttagtagaattaattttgaagaattatataatgttatcattttaccatttaataaaattaaattccaacaaacagaggaacctgttgatatcaatttagaacatactactttacaaccatatattaataaacaactaggtattgattccaaaaaaacagggatatatagagcaccaagatctactggtctagttaacttaaatggatatctttcttcaaaacttccagatagggaaaaatcttccttacctcctggatgtaattttgttaatgaaaataaaaattataataaaaagaaaaaaaaaaagagaagttcaaaaagtaaacaaaagagagaaacattctaa

SEQ ID NO: 8 - DNA, 8625 bp, Plasmodium falciparum
  misc_feature (1)..(8625): PFD1060w
atggctgaggaatatgagaaatttaagcgatttgaatatcggatgaactccaaccttgttttacaaagggaaggtccaatatcgaacaccaaagaacccaccggtgaaagcgagagtttagtaggaagattaaaatataagatgggtgataaggttgaatatagtaataagaataagaagaatattatgttagtacaagataataaagaaatggctataaatagaaataaaagaaaagatatttttgatgatgataaatataatagacgatctaataagaaaattcgtcataaagaaaagagtgtattaaatgtaaacattgaagatatttttttatataagcctacaacaaagtatacagaagagatatatacgaaacttatgagtaagataagatttttattaggtgataacacaggagatataattaatagtgcttgtaacgaaatcttatacatattaaaaaatgaagaattaaataatgaagagaaaaagaaacaggtggaaagtgaactagaaatttatataaatgatgatatatttattgaaataaataatttatcgaaagaaatatatgattttaataaacaagaagaaggggaatatgtagaaaatgacgagggtgttgccgttatttttgaagaagatgatgattattttaatataggaaggaatattaccaggggatacataaatgatcacgagactatggaattacaagaattgagtgatgataatgaggaggaggatatttctgaagatgatgatggagatgatgaagaagaagatgatgaagaagcagatgctgaagaagcagatgatgaagatggagatgatgaagatggagatgatgaagaaggagatgatgaatatggagagaatgaagacggagatgataacaataataataataataataataataatagtgtgagaagaaaaaagaaaaagaaaggtaaccaaataaaccatctcaaaagttatgataaaaaaaataagaaattcgaaaattatttaagtttaaaaaatacaaataagaacttgtattatgatgaagaaaataaaaatgttcaagataatgatgaacttaatattaacgttatcgattcacattggttacaaagagaattaaataaaatatttccagatccttctctatgtttagataaggaaaaagaagttttaaatgttttaaatatatatgatatacaagaaagtgagaataaattaatgcatatattaaaatatgaaaattttcatattgcaagagtgttaataaaaaatagatggaaaatatattattgcaccttactcggtcaggcacaaacggaagaagaaaaagaagaaataaaaaaggagatgaaaaagacagaagaggggcaagatattttagatgaattatgtaactttaaaaatgttagaaagaataaacaaagtgaatttagtaaaataataaggaaagaagctgataatttgtttggaaagaaaaaaataaatgattataataaaaaaggaaacatgtcagattatacatatatacatgatgatgatgataatgatgataataatgatgataataatgatgataataatgatgatgataataatgatgatgatgatgaagaaaaggaggagggtcctaaatattatgacgaggaaaatgttagtgatgatgataattattacgaatctacaaagttgtataatagcgattcgcaaggagaagacgcaaataaaaagataaatataaatgcaaaagatgtaaaaaatataaagggtgtaaaagaagaagataatgatggaggatcggggaaagaagataatgaacctacacagaaaagtttttataataatatgaaatataaatttatcgatttagaaaaactagaaacaaaagaaaagaataaagatatattttttaataaagaagtaatattaccaccagaaagtaaaagaatagaaagaaaagaatatgatgaaataattattagtagtatgaaagataataaaatggatggtatgaaaagaacgaaaaataataaaatgaattattatacatctccagaagaaataaaattgatacatataaatgaattacctgagtggacacatgaagtattttcttgtgttaatattactaaattaaatccaatacaatcaaaagtatatgatgtggcatttaataaatatgaagaaaatatgttaatatgtgctcctacgggttcaggaaaaaccaatattgctttattatgtattttgaatgttattaattcatatcgattaacaagtgggaacataaatagaaaggattttaagattgtatacatatcacctatgaaggctttggtaaatgagcaggtgcaatcatttaatctacggctcaaatgtatgaatattaaggtgagtgaattaacaggtgacgtaaatttaagtactaaagaattggacgatagtcaaattattgttatgacgcctgaaaaatttgatgttattagtagaaaatggaatgaaaagatattattgcacaaaataaaattgattatatttgatgaaatacatttgttgaatgagatgcgaggaaatgttttggagagtatcatagcacgtataaatagatatatggataacacgatggtatatgatatgggtgacgttgcggatggttcatctggaggtatccgaggcaaaaataaagatgctaataggtataataataatgatgataaaaaaaatgacgataataataatgatgataaaaaaaatgacgataataataatgatgataaaaaaaatgacgataataataatgatgatgatgacaattataatggtggtggtgttaattctgttcgacagaataagtatggagaaaaccatatatcgataagaaggaaaaagattcgattggttggtttgtctgctacgttaccaaattatgaagatgtaggtatatttttaagagctcatatcgagaggggtatattttattttgatcattcctttagaccagtacaaattgaacaacattatataggtataaaagaaaagaaaggaattaagaagtatgctcttatgaatgagttaacatatgagaaagtacttgaagaagctggaaagaatcaaatattaatttttgttcatagtagaaaagaaacatatagaacatcaaaattgttgattgatagatttatgaaaagcgataacttaagtaaatttttaatagataagaaaatatctagtgaaatattattatcggaaaaagaacatgtaataaatgaagaattaaaagagattttaccatttggatttggaattcatcatgcaggattaaaaagattagaccgaaaattggttgaagatttattttctgatagacatatacaagttttagtttgtacaagtactttagcttggggtattaatttacctgcgcatacagtaataattaaaggtacgagtgtttataacattaatataggagattttgatgaattatcgagtatggatgttttacaaatggttggacgttcaggtagaccacaatatgataagagtggtaaagctattataataacagatcataagaatttacaattatatttatctttaaacaacgaacaattatttatagaatctacattgcttcataatattgttaatattataaatgccgagattgttttaaaaaatattcagaatatggatgatgcgatgaattggttggagcatacatatatgtatatacgtatgttaaaatgtcctagtttatatggtgtgataataaattcgaatgataaaattaaaggtattgaagaatttaaagatattatagatatgaataataaaacaaagaaacataataataataataataacaatataaatggtggtaataataataataataacaatataaatggtggtaataataataataataacaatataaatggtggtaataataataatagtaataatgttatggattatatgtccattttaaaaacaaattataagtataataaatttatggaaaaaataaaaagaaggatgtacaatatattatattcatgttttcttatattagaaaaatatgatttaataaaatataataaaaaattaaatacagttagtagtacatatataggtaaaattagtagttattattatgttgattataaatctatagatatatataataaaaaattaaataaatatacgaatgaaatagatttattaaaaatatttactatgagtaatgaatttaaaaatatatttataagagatgaagaaaaaacagaattgtcaattataatggaaaaattaccaatacctgttaaggaatctataaatattccttatacaaaaataaatatattattacaattatatttatcgaatataatattgaatggttatattataaatgcagatatggtatatatacatcagaatgcattacgtatttttagatcattttttgaaatatccttaaagaaaaattcttataatttaataaaattaactttaaagttttgtaaaatgattgaaagaaggatgtggagtactatgacaccattaagacaatttggtttattaagtactgaacttatacgaataattgaaaaaaaaaatattacattcaaaaattatttaacaatgaatttaaatgaatatataacaatttttaaaaataaaaaaattgcaaaaaatatatataaattagtacatcattttccaaagttagaattaaatgcatatatacaacctattaatcataaaatattaaaagttgatttaaatatagcaccagattttatatataacccgaaatatcatggatattttatgttattctgggtatttgtttttgatatatcaaatgaaagtatattacattatgatttatttacactcaaaaaaaattataaaaatgatatcctaaaccaaacacaaagatataacaacaacggcaataatagcaatatgtataacccatcagatgttttagatgatcatgtgttgacatttttcttaccaataaatgataatcctttctatattgtcaaagtcatttcagataaatggctagaatgtgaagctaccataaatttatatctaaaagatttaattcttccctcgcaaaatttttattctacacaattgttggatctacaacctttgccaatacattcaattaaatatgacaaggcacataccttttttaataatataagaaatttacattattttaattctatacatacacaaatattttcatctatttttgaaaataacggaaatgtactaatagctagctcaaattcaaaacattatttaattccagccgaattaggtatgataaaaatcttgaaattcttacattgtttttataatttcatcaacacgtatattaaaaaagaaaaagatgtatataaaattattaatgataaaaagctagctgatcttttatataataataatttaatagatctaataaaaattgtgtatatagcaccattagatgatgtaattataaaaacatttaaaaattggcaagccatgaaaaaaacatttgatttgaaaatgtgtatactaacaggagatatacaaatagatatgaagctattacaaactcatcatattattttatccaacccatccaattatgataacatatcaaaaaaatggaggaggaaaaaaatcctccaaacggtgtctttttatatatttgatcatatggaattattggatacggtacaagggggaatcatggagatattaataagtagaataagatatatatcgacacaattaaatataaataaatcagaaaaaaaaaaccaacacaataatgataataataataatgataatttaatcatgtcgatattaaatatagatatgatcaaaatggataatgtagaagatgataataataagaaaaatataatacataacaataataataataatagtaatagtaagaaggatgattttgtacaaactataaatgcattacaaaattttactattgacaatatttatgattatataggattaaatagaatattgtgcctatctagttgttccttatataattcaaaagatttcgctgaatggatcggatgtaaaaaaaatgattattataactttttatcaacggttagagatatacctattgaaatatacttgcatgcagttaatattatgaataaacaaaatagatatatatccatgcaaagacaagtttatcaaaatataagaaaattaaagaatataccaaaaaaaaaaaaaatgcaaaatgttattatttttgtaacagatcaaaaattgtgtaaaacattggcattagatttaatcctttcagcttataatgacaattttaaatatttttcgctttttgaaaattatgaagatgataaatatgacacatccaaatcttctactaatccatatgatcctaagaatgaagaaaaagaaaagaaaaaaggaggagggtggatatccaatttgttcacctacaacaaagatgataaaatcggtcgtcataacgaaagcaatgatttaataaataacaattcacatgatgatacaacatcaaataatttacaaaaatctggaacatttaggagtaatgatttatataataatggtaccaatgtggaggaactagctaaaagtattcataatatgattaatttgaatatatccaatatggatattattaagaataaggaagtagcagaaaataacatgaatgttgcaagtagtaataataataataataataatagtaatagtaataatatttataaaaatgggcggaaacgttctaataatagaaataataagaatgaagaacatgtatttgattatattaatgataaaatgttaagacaatttatgaaaaaaggtatttgctatttacataataatatgacagaaattgaaaagaaaattgtggaaatattatttgataaaaaaaccatacaaatattaatcgtttcttatgattatatttacagtttaaatgtttatgcaaataatgttattatattagatactattataacacatttccataacaataaagaagaagattatagtatacaaaatatattagaaatgataagctatgctggacgacaaaatgaagataccaaagcttttgtatatatatatacatatataactaaaaaagaatattataaaaattttatatatgaaccattaactgtagaatctaatattgaagataacttaccaaatttcctaaataatgaaattgttatgagtactatagaaaattatcaagatgctatcgattggttaacatggtcctttttttatagacgtataaaaaaaaatccaaattattatggtttaaaaggtatatccaatgaacatatatcagattatttatcggaacttatagaaaataatatggaaatattatcatttgctaattgtataaatatagaagaacaaactatggatattaaaccatgtaatttgggtattatatcatcattttataatttagattatcatattattcatttttttaatcaatatgtattatctttaaaaacattaaagaaatcaagaatttttgaaattatatgtctttcgaatatttttaatgacatattgaaaatacataattatgatatatttctatgtttaaaaattgctcaagcatgtaataatgtacaagtaacatatgagtttttaaaactttctattaataatgaaaatacattaaaaaatgcaaatattgaagataatttaaataaagatacgaaatcagaagattataaaaaagatcaatatattaatttattacaatttatgagtgtacctatgtattttacatctcatttaaaagcttttatacttttacaagcacatattcatagatatagtttacctctgaattatatacaagaaacaaaaactgtattattaaaagcatataaattaataaattccttaattgatgttataagtagtaataatattttaaatttttgtttatttgttatggaggtctcacaaatgttaacacagagtatgaagagtactgatcaatccaatttatatcaattaccacattttgatgaacacttaattaaaaaagcaaacgatttagaaatattggatgtgtatgatttaataaatgctgaagatgaaccaagagatattttattaaaacatctgaatgaaaaacaaagaagtgaaatcgcaaatgtttgtaatatttttccaattattgaagtacaatatgaaatcgatcttgataaatcttataaagttaatgaaatagctcaacttaatttaaccatagaaagagatttaacagatgatgctgtgatttttgcacactcactttatttaccatttgaaaaagaagaaatgtggtggatcgttatcggaattaaaaaaatgaacttacttttatctatcaaaaaattatctttattgaaaagtgtcaataatataaaaattaatttcgaattgcctgacaaaccaaatacctatgacgtagtaatttatgtcatcaatgattgttatgttgggtgtgaccaggagtacgagtttaagattaacgtggaggagtaa